SEQUENCE LISTING (26 sequences)

SEQ ID NO: 1 - DNA, 20 bp, Unknown
  ACTIN forward primer 5′ to 3′
ggcatcctcaccctgaagta

SEQ ID NO: 2 - DNA, 20 bp, Unknown
  ACTIN reverse primer 5′ to 3′
ggggtgttgaaggtctcaaa

SEQ ID NO: 3 - DNA, 19 bp, Unknown
  SOX2 forward primer 5′ to 3′
cccagcagacttcacatgt

SEQ ID NO: 4 - DNA, 20 bp, Unknown
  SOX2 reverse primer 5′ to 3′
cctcccatttccctcgtttt

SEQ ID NO: 5 - DNA, 20 bp, Unknown
  OCT3/4 forward primer 5′ to 3′
cctcacttcactgcactcta

SEQ ID NO: 6 - DNA, 20 bp, Unknown
  OCT3/4 reverse primer 5′ to 3′
caggttttctttccctagct

SEQ ID NO: 7 - DNA, 20 bp, Unknown
  KLF4 forward primer 5′ to 3′
gaccacctcgccttacacat

SEQ ID NO: 8 - DNA, 20 bp, Unknown
  KLF4 reverse primer 5′ to 3′
tgggaacttgaccatgattg

SEQ ID NO: 9 - DNA, 20 bp, Unknown
  C-MYC forward primer 5′ to 3′
aagaggacttgttgcggaaa

SEQ ID NO: 10 - DNA, 20 bp, Unknown
  C-MYC reverse primer 5′ to 3′
ctcagccaaggttgtgaggt

SEQ ID NO: 11 - DNA, 20 bp, Unknown
  NANOG forward primer 5′ to 3′
ctccatgaacatgcaacctg

SEQ ID NO: 12 - DNA, 20 bp, Unknown
  NANOG reverse primer 5′ to 3′
ggcatcatggaaaccagaac

SEQ ID NO: 13 - DNA, 20 bp, Unknown
  LIN28 forward primer 5′ to 3′
cacagggaaagccaacctac

SEQ ID NO: 14 - DNA, 20 bp, Unknown
  LIN28 reverse primer 5′ to 3′
tgcaccctattcccactttc

SEQ ID NO: 15 - DNA, 20 bp, Unknown
  UTF1 forward primer 5′ to 3′
agctgctgaccttgaaccag

SEQ ID NO: 16 - DNA, 18 bp, Unknown
  UTF1 reverse primer 5′ to 3′
gtgggaaggcagcaggag

SEQ ID NO: 17 - DNA, 20 bp, Unknown
  ABCG2 forward primer 5′ to 3′
agctgcaaggaaagatccaa

SEQ ID NO: 18 - DNA, 20 bp, Unknown
  ABCG2 reverse primer 5′ to 3′
tccagacacaccacggataa

SEQ ID NO: 19 - DNA, 20 bp, Unknown
  REX1 forward primer 5′ to 3′
tttacgtttgggaggaggtg

SEQ ID NO: 20 - DNA, 20 bp, Unknown
  REX1 reverse primer 5′ to 3′
tctgttcacacaggctccag

SEQ ID NO: 21 - DNA, 20 bp, Unknown
  DNMT3B forward primer 5′ to 3′
ctactgcccagcatgtcaga

SEQ ID NO: 22 - DNA, 20 bp, Unknown
  DNMT3B reverse primer 5′ to 3′
ttgcacccaggatccttaac

SEQ ID NO: 23 - DNA, 20 bp, Unknown
  TP53 forward primer
ggcccacttcaccgtactaa

SEQ ID NO: 24 - DNA, 20 bp, Unknown
  TP53 reverse primer 5′ to 3′
gtggtttcaaggccagatgt

SEQ ID NO: 25 - DNA, 20 bp, Unknown
  hTERT forward primer 5′ to 3′
gccgtttccaaacacagagt

SEQ ID NO: 26 - DNA, 20 bp, Unknown
  hTERT reverse primer 5′ to 3′
aacacctagcatgggtgagg

(19) United States
(12) Patent Application Publication
(10) Pub. No.: US 2013/0266541 A1
(43) Pub. Date: Oct. 10, 2013

(54) HUMAN INDUCED PLURIPOTENT STEM CELLS

(71) Applicants: Elias T. Zambidis, Baltimore, MD (US); Tea Soon Park, Baltimore, MD (US)

(72) Inventors: Elias T. Zambidis, Baltimore, MD (US); Tea Soon Park, Baltimore, MD (US)

(73) Assignee: THE JOHNS HOPKINS UNIVERSITY, Baltimore, MD (US)

(21) Appl. No.: 13/441,328

(22) Filed: Apr. 6, 2012

(51) Int. Cl. C12N 5/10; A61K 35/12

(52) U.S. Cl. 424/93.7; 435/373; 435/325; 435/366

(57) ABSTRACT

The present invention relates generally to the field of stem cells and, more particularly, to reprogramming blood cells to pluripotent stem cells. In a specific embodiment, a method for producing an induced pluripotent stem cell from a human myeloid progenitor cell comprising the steps of (a) activating the human myeloid progenitor cell by incubation with hematopoietic growth factors; (b) transfecting the activated progenitor cells with a non-viral vector expressing one or more pluripotency factors; and (c) co-culturing the transfected cells with irradiated mesenchymal bone marrow stromal cells.

### STATEMENT OF GOVERNMENTAL INTEREST

This invention was made with U.S. government support under grant no. U01 HL099775. The U.S. government has certain rights in the invention.

### FIELD OF THE INVENTION

The present invention relates generally to the field of stem cells and, more particularly, to reprogramming blood cells to pluripotent stem cells.

### INCORPORATION-BY-REFERENCE OF MATERIAL SUBMITTED ELECTRONICALLY

This application contains a sequence listing. It has been submitted electronically via EFS-Web as an ASCII text file entitled “P11206-02_ST25.txt.” The sequence listing is 5,107 bytes in size, and was created on Mar. 29, 2012. It is hereby incorporated by reference in its entirety.

### BACKGROUND OF THE INVENTION

A major limitation of the clinical utility of human induced pluripotent stem cells (hiPSC) is their high propensity for malignant transformation. This risk for clinical cell therapy is substantial with the use of retroviruses and lentiviruses for expressing reprogramming factors because of their tendency for random insertional mutagenesis. The potential for malignancy is theoretically reduced via reprogramming with fewer integrated factors. However, despite the overall tendency for silencing of integrated viral vector promoters, low levels of reactivating transgene expression of these proto-oncogene factors remains problematic. For example, chimeric mice made from iPSC generated with virally-expressed pluripotency factors eventually formed malignant tumors, even in the absence of ectopic Myc expression.

Safer methods for generating iPSC from somatic cells which greatly reduce these risks avoid the use of stably integrating sequences, and employ the use of non-integrating episomal DNA vectors (e.g. adenoviral or EBV-based plasmids), repeat transfections with plasmids, secondary excision of integrated transgenes, and direct transduction with pluripotency factor proteins. Among these methods, those that use downstream excision of transgenes (e.g. Cre-loxP and piggyBac transposition) are reasonably efficient, but continue to risk harmful genomic recombination, and leave potentially harmful residual viral elements in the genome. Non-integrating nucleic acid transfection and direct protein transduction are theoretically the safest approaches, since they do not leave permanent genetic footprints. However, these methods are currently extremely inefficient, technically burdensome, and produce only rare reprogrammed iPSC. Additionally, recent studies have reported that hiPSC derived with viral vectors from fibroblasts may have deficiencies in their ability to differentiate into therapeutically relevant lineages, or serve faithfully in disease modeling compared to human embryonic stem cells (hESC). Such iPSC may be partially reprogrammed, or have incomplete transgene silencing. It is currently unknown whether hiPSC made with alternative non-viral approaches will have similar, or fewer limitations for generating therapeutically relevant cell lineages.

### SUMMARY OF THE INVENTION

The present invention is based, at least in part, on the development of an optimized system for generating non-integrated, virus-free human iPSC from ex vivo mesenchymal stroma cell (hMSC)-activated CD34+ cord blood (CB) progenitors using non-integrating factors. In contrast to the low efficiency of non-viral iPSC generation from fibroblasts or keratinocytes, hMSC-primed CB CD34+ progenitors were rapidly and fully reprogrammed with non-integrating plasmids. Reprogramming was at least 300 times more efficient than has ever been reported for any human non-viral system, and correlated to high endogenous expression of a core ESC-like transcriptome in CD34+ progenitors. Low passage CD34-iPSC subclones were vector and transgene-free, possessed molecular signatures that were highly similar to hESC, and differentiated robustly to vascular, hematopoietic, neural, and cardiac lineages. The present invention shows that CD34+CB progenitors represent a superior somatic source for generating high quality, clinically safe iPSC that are more akin to hESC.

Accordingly, in one aspect, the present invention provides methods for producing an induced pluripotent stem cell from a human myeloid progenitor cell. In one embodiment, the method comprises the steps of (a) activating the human myeloid progenitor cell by incubation with hematopoietic growth factors; (b) transfecting the activated progenitor cells with a non-viral vector expressing one or more pluripotency factors; and (c) co-culturing the transfected cells with irradiated mesenchymal bone marrow stromal cells. In a more specific embodiment, the method comprises (a) activating the human myeloid progenitor cell by incubation with hematopoietic growth factors; (b) transfecting the activated progenitor cells with an episomal plasmid expressing one or more pluripotency factors; and (c) co-culturing the transfected cells with irradiated mesenchymal bone marrow stromal cells.

In a specific embodiment, the human myeloid progenitor cell is selected from the group consisting of cord blood cell, adult bone marrow cell and adult peripheral blood cell. In certain embodiments, the human myeloid progenitor cell is a cord blood cell. In a more specific embodiment, the cord blood progenitor cell is CD33+CD45+. Alternatively, the cord blood progenitor cell is CD34+CD38+.

In particular embodiments, the hematopoietic growth factors comprise Flt3 ligand (Flt3L), stem cell factor (SCF), and thrombopoietin (TPO). In other embodiments, the one or more pluripotency factors comprises sex-determining region Y HMG box 2 (SOX2), octamer binding transcription factor 4 (OCT4), Kruppel-like factor 4 (KLF4) and v-myc myelocytomatosis viral oncogene homolog (MYC). The one or more pluripotency factors can further comprise NANOG, LIN28, and simian virus 40 large-T antigen (SV40LT). In a specific embodiment, the one or more pluripotency factors is selected from the group consisting of SOX2, OCT4, KLF4, MYC, NANOG, LIN28, and SV40LT. In certain embodiments, the transfection method is nucleofection.

In other embodiments, a method for producing an induced pluripotent stem cell from a CD33+CD45+ cord blood progenitor cell comprises the steps of (a) activating the cord blood progenitor cell by incubation with Flt3L, SCF and TPO; (b) nucleofecting the activated progenitor cells with an episomal plasmid expressing SOX2, OCT4, KLF4, and MYC; and (c) co-culturing the nucleofected cells with irradiated mesenchymal bone marrow stromal cells.

In another embodiment, a method for producing an induced pluripotent stem cell from a growth factor activated human myeloid progenitor cell transfected with an episomal plasmid expressing one or more pluripotency factors comprises the step of co-culturing the transfected cells with irradiated mesenchymal bone marrow stromal cells following transfection. In such embodiments, the human myeloid progenitor cell is selected from the group consisting of cord blood cell, adult bone marrow cell and adult peripheral blood cell. In a specific embodiment, the human myeloid progenitor cell is a cord blood cell. In a more specific embodiment, the cord blood progenitor cell is CD33+CD45+. In an alternative embodiment, the cord blood progenitor cell is CD34+CD38+.

Furthermore, in such embodiments, the one or more pluripotency factors comprises SOX2, OCT4, KLF4, and MYC. The one or more pluripotency factors can further comprise NANOG, LIN28, and SV40LT. In certain embodiments, the transfection method is nucleofection.

In another aspect, the present invention provides induced pluripotent stem cells. In a specific embodiment, an induced pluripotent stem cell comprises an episomal plasmid encoding SOX2, OCT4, KLF4, and MYC, wherein the induced pluripotent stem cell was co-cultured with mesenchymal bone marrow stromal cells following transfection with the plasmid. In another embodiment, an induced pluripotent stem cell comprises an episomal plasmid encoding SOX2, OCT4, KLF4, MYC, NANOG, LIN28, and SV40LT, wherein the induced pluripotent stem cell was co-cultured with mesenchymal bone marrow stromal cells following transfection with the plasmid. In such embodiments, the pluripotent stem cell was induced from a human myeloid progenitor cell. The human myeloid progenitor cell can be selected from the group consisting of cord blood cell, adult bone marrow cell and adult peripheral blood cell.

In certain embodiments, the pluripotent stem cell was induced from a cord blood progenitor cell. In a more specific embodiment, the cord blood progenitor cell is CD33+CD45+. In another specific embodiment, the cord blood progenitor cell is CD34+CD38+. In certain embodiments, transfection method is nucleofection.

Certain embodiments further provide an enriched population of isolated pluripotent stem cells produced by a method of the present invention. In such embodiments, the isolated pluripotent stem cells express a cell surface marker selected from the group consisting of SSEA1, SSEA3, SSEA4, TRA-1-60 and TRA-1-81. In other embodiments, the isolated pluripotent stem cells express high embryonic stem cells (ESC)-like levels of MYC and OCT4-associated circuits and inactivated ESC-like Polycomb group (PcG)-regulated networks. In a further embodiment, a method for treating a disease requiring replacement or renewal of cells comprising the step of administering to a subject an effective amount of the pluripotent stem cells of the present invention.

### BRIEF DESCRIPTION OF THE FIGURES

FIG. 1 shows that bone marrow stromal cell (BMSC) co-culture primed a highly efficient non-integrated bulk reprogramming of cord blood (CB) progenitors that required only four episomal Yamanaka factors on a single plasmid. The role of a brief 3-day BMSC co-culture of CB CD34⁺ progenitors following a single pulse of nucleofected episomal plasmids on Day 0, and the number of episomal factors required for efficient reprogramming was quantitated, as described in the Examples section. The experimental design is summarized in FIG. 9. In FIG. 1a, soluble factors from brief BMSC co-culture preserved the viability of OF-activated CB progenitors. Viable CB cells were enumerated via Trypan Blue on Day 3 following nucleofection with four episomal factors (SOX2, OCT4, KLF4, MYC; 4F) on Day 0 (see experimental design in FIG. 9). As shown in FIG. 1a are the fold-increases of CB cell numbers from Day 0 to Day 3 that resulted from: no BMSC co-culture (−BMSC), with irradiated BMSC co-culture (+BMSC), or with BMSC co-culture but separated by a Transwell insert (BMSC(T)). The Transwell culture well insert prevented cell-cell contact between MSC and CB cells, but allowed soluble stromal factors to diffuse freely to the cultured CB cells. Averages, SEM, and p values (t test) of n=4 averaged experiments is shown. NS=not significant.

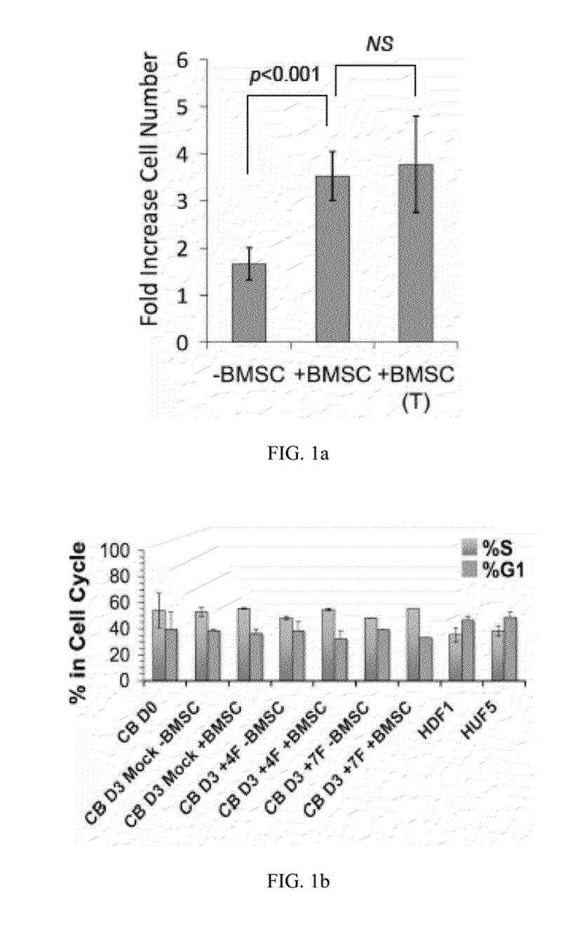

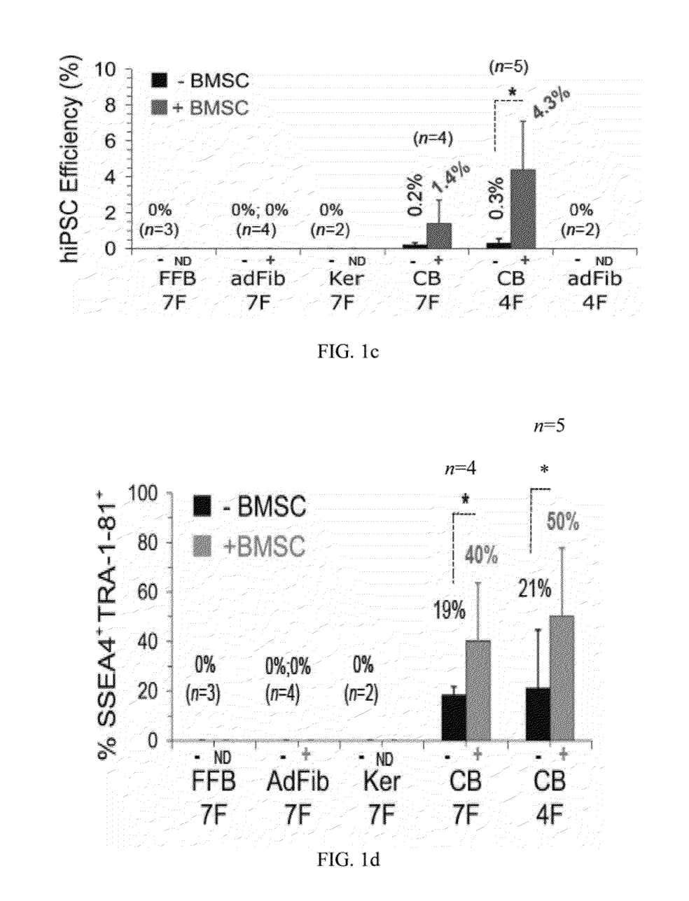

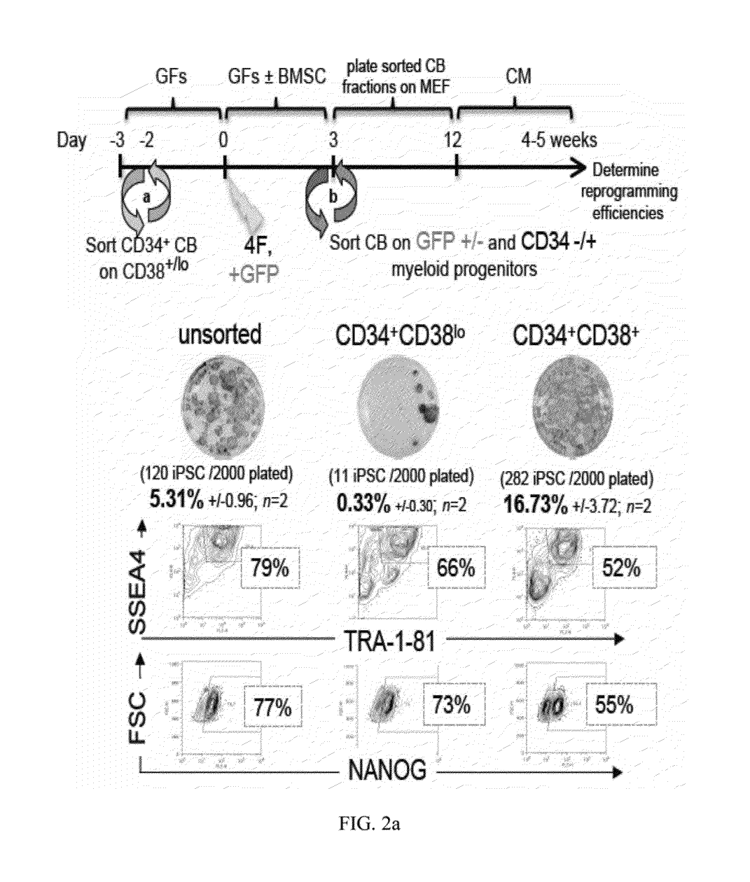

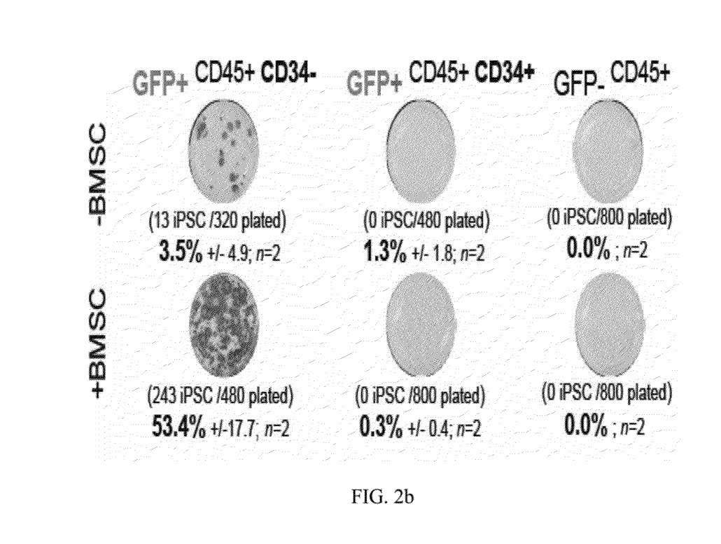

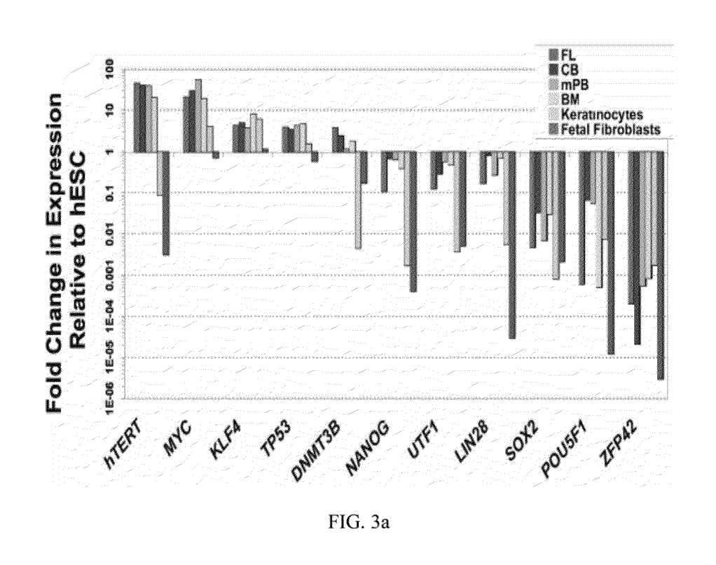

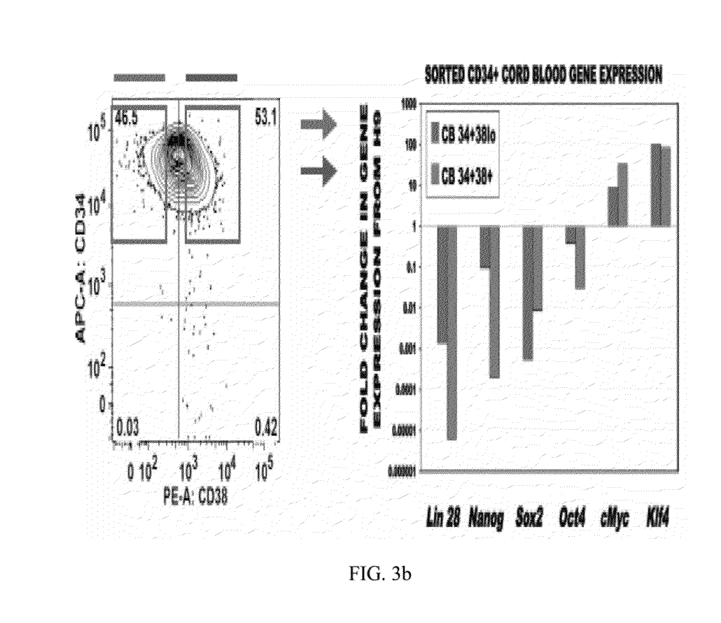

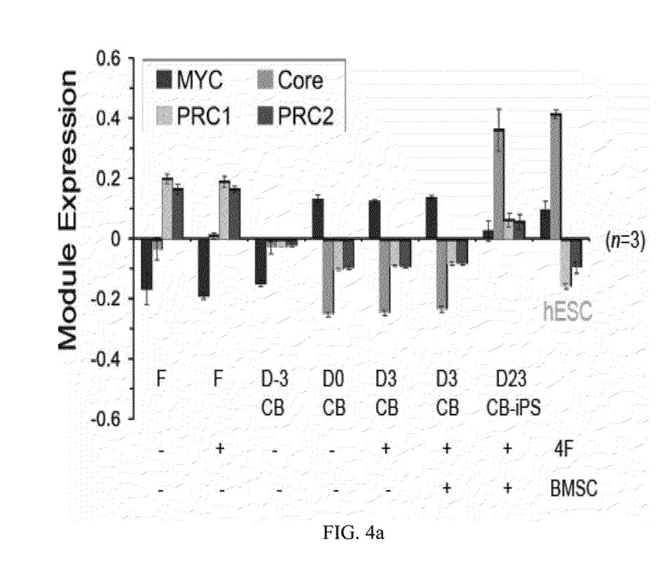

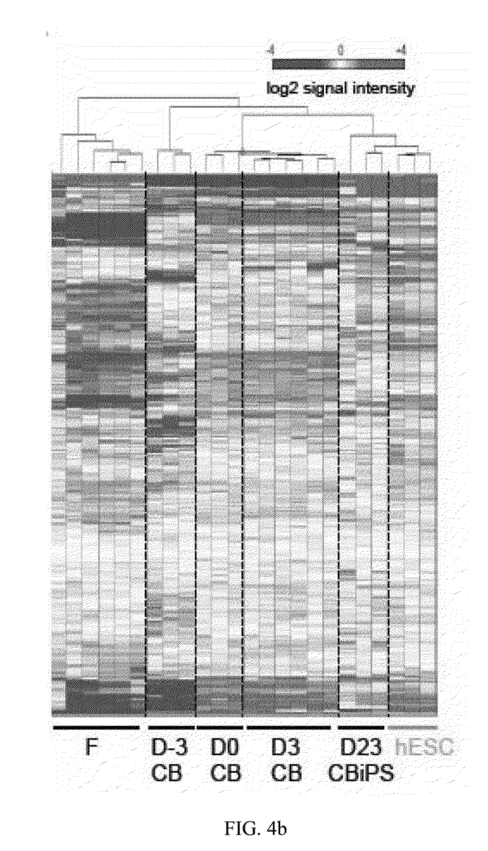

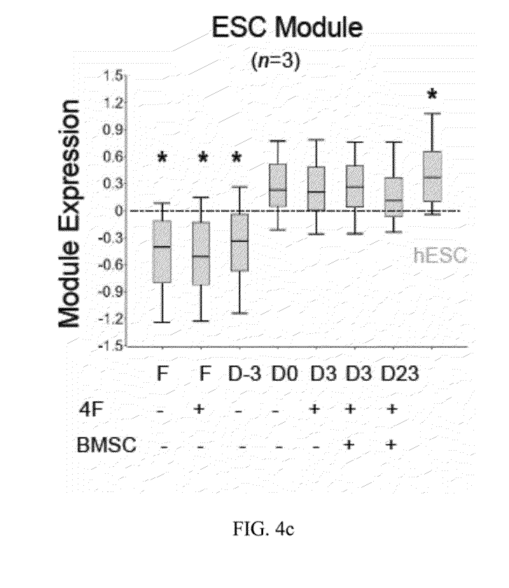

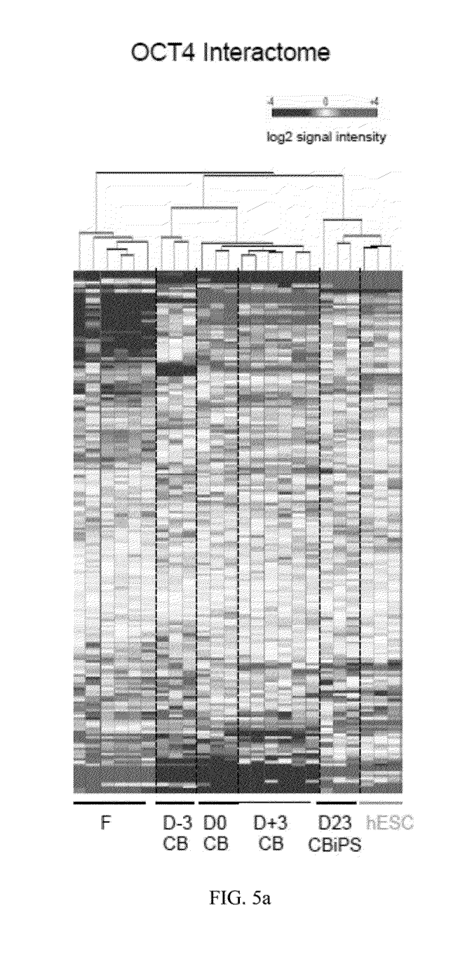

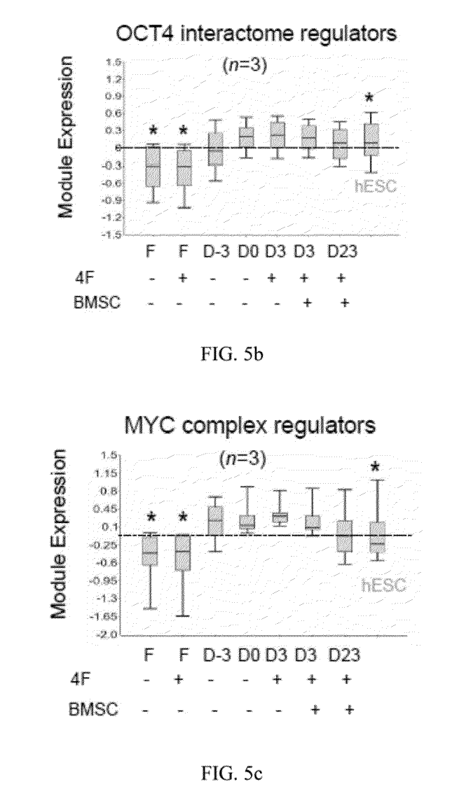

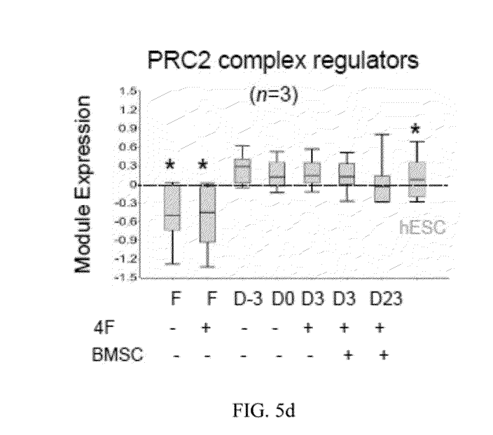

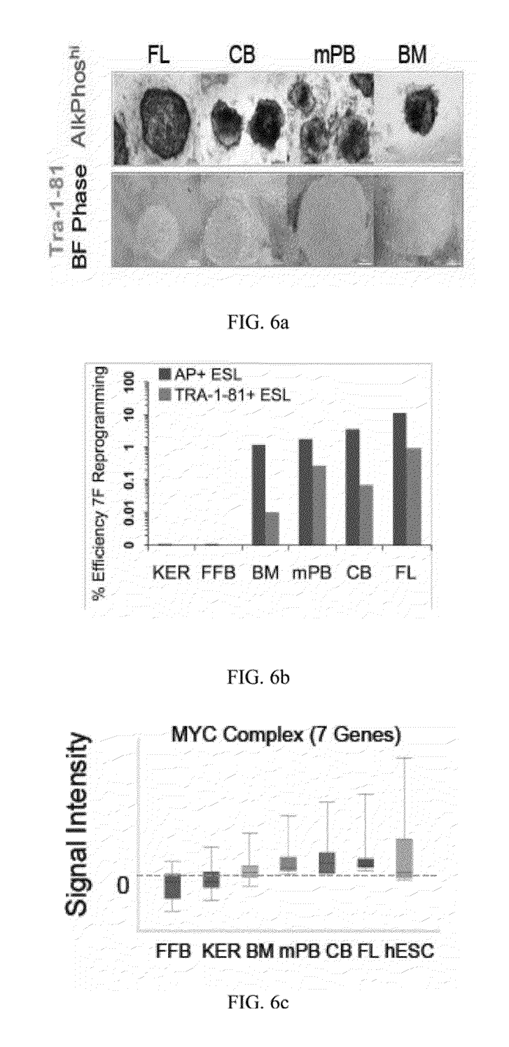

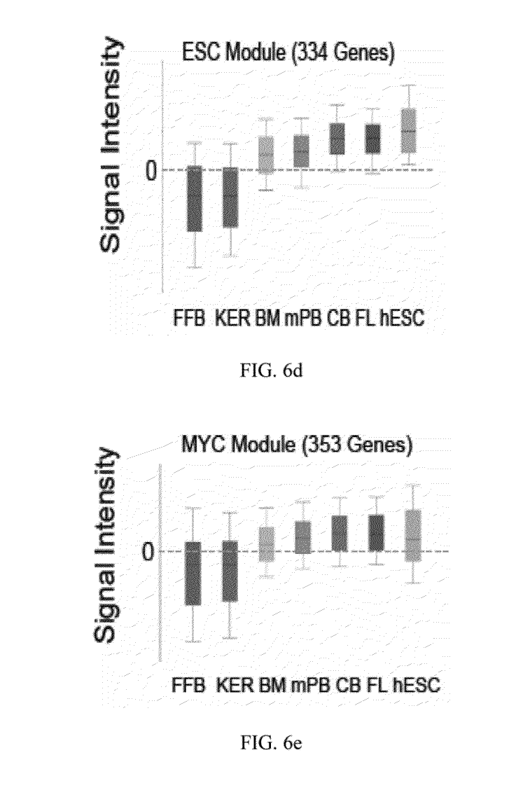

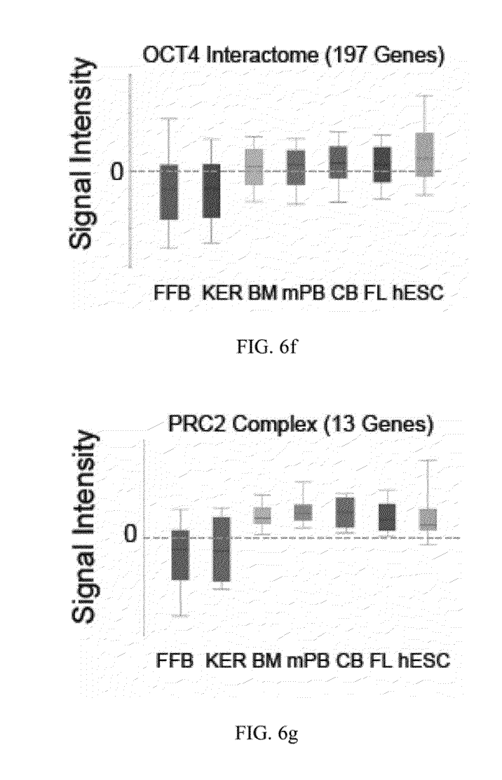

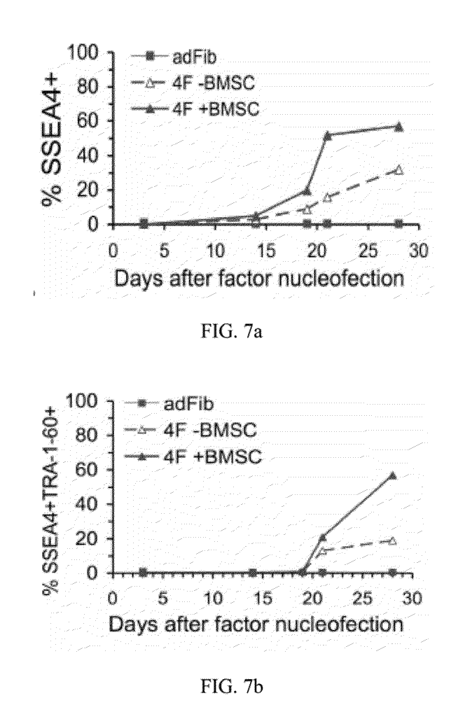

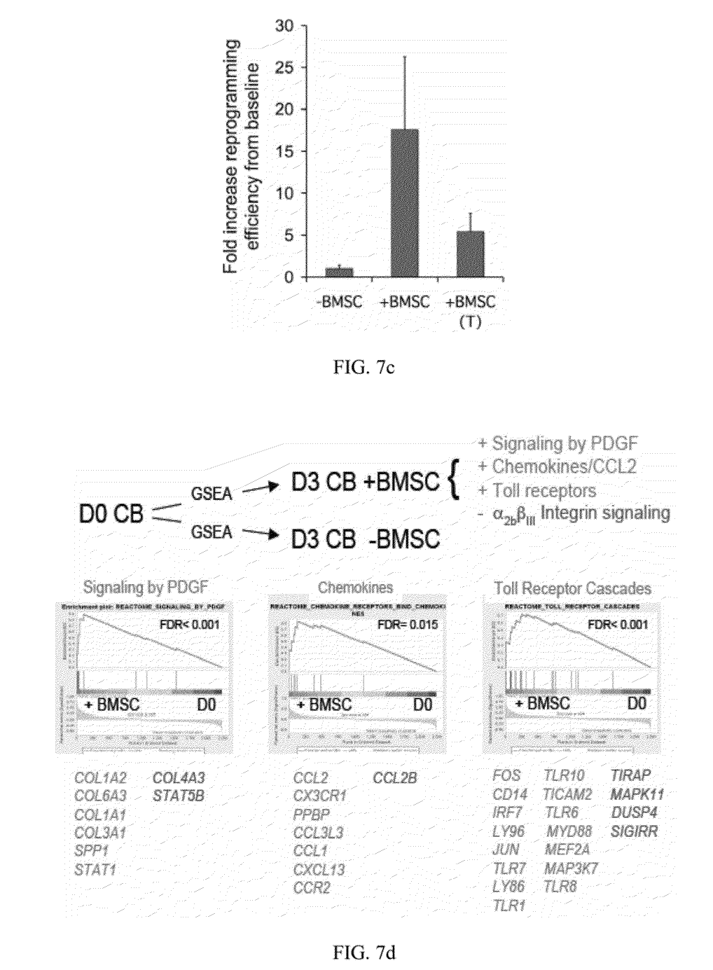

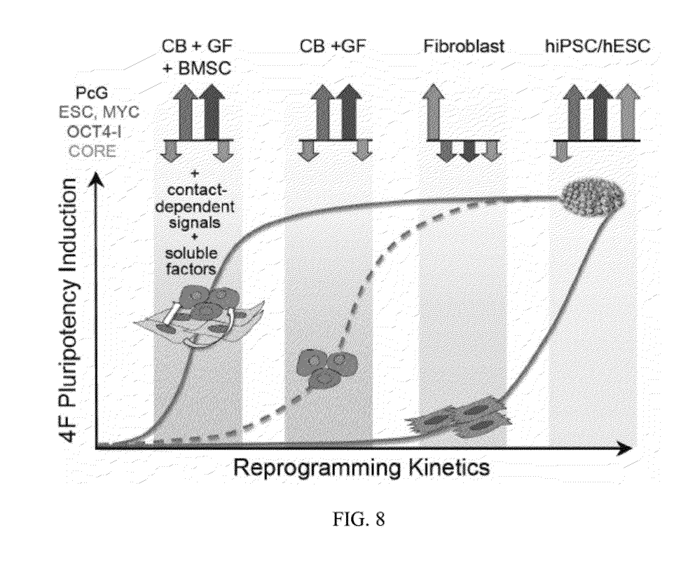

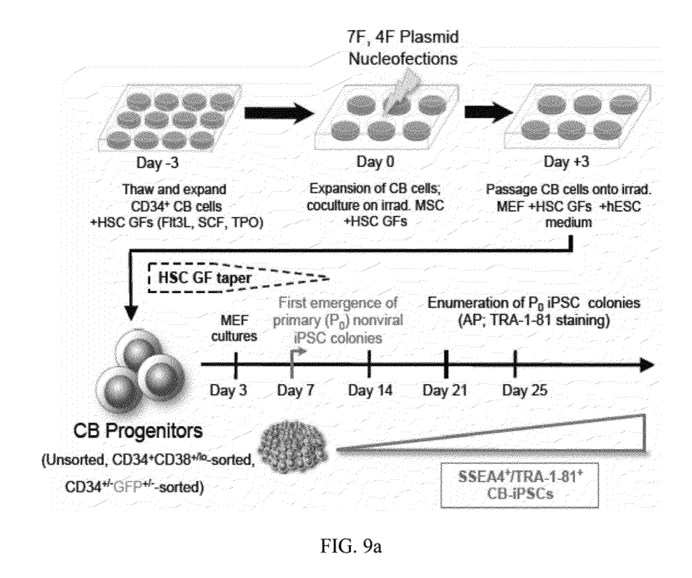

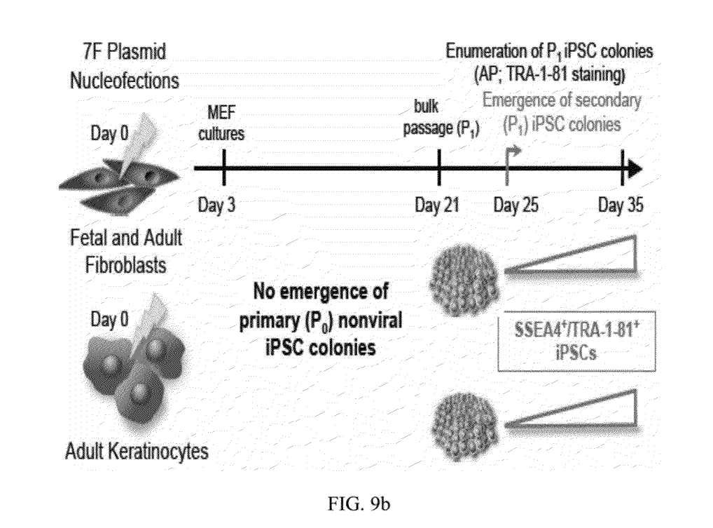

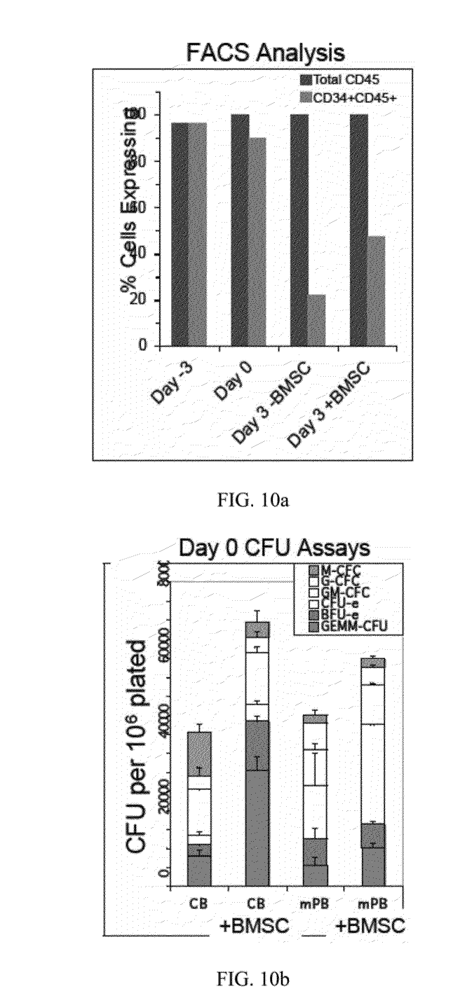

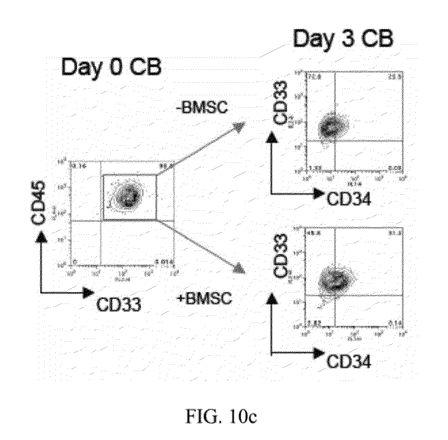

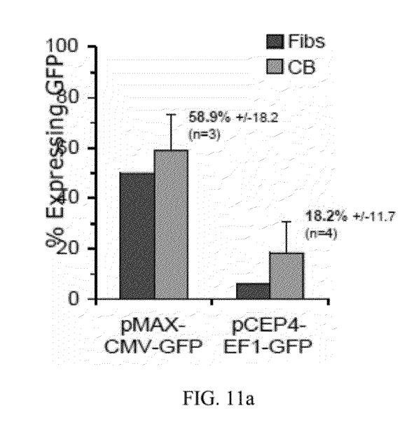

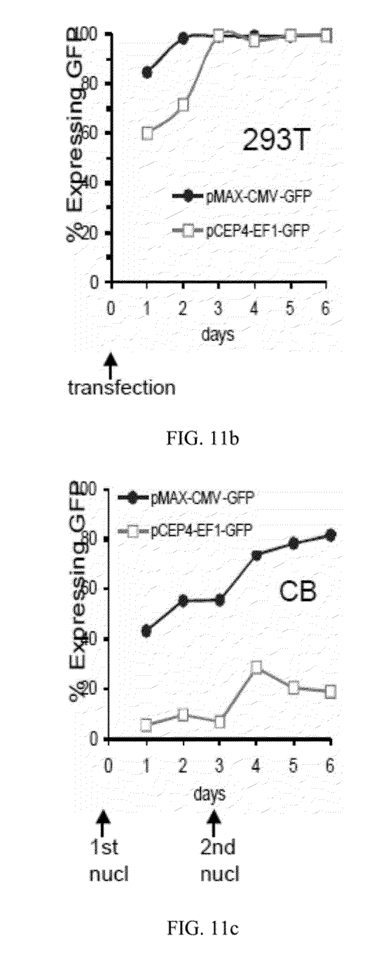

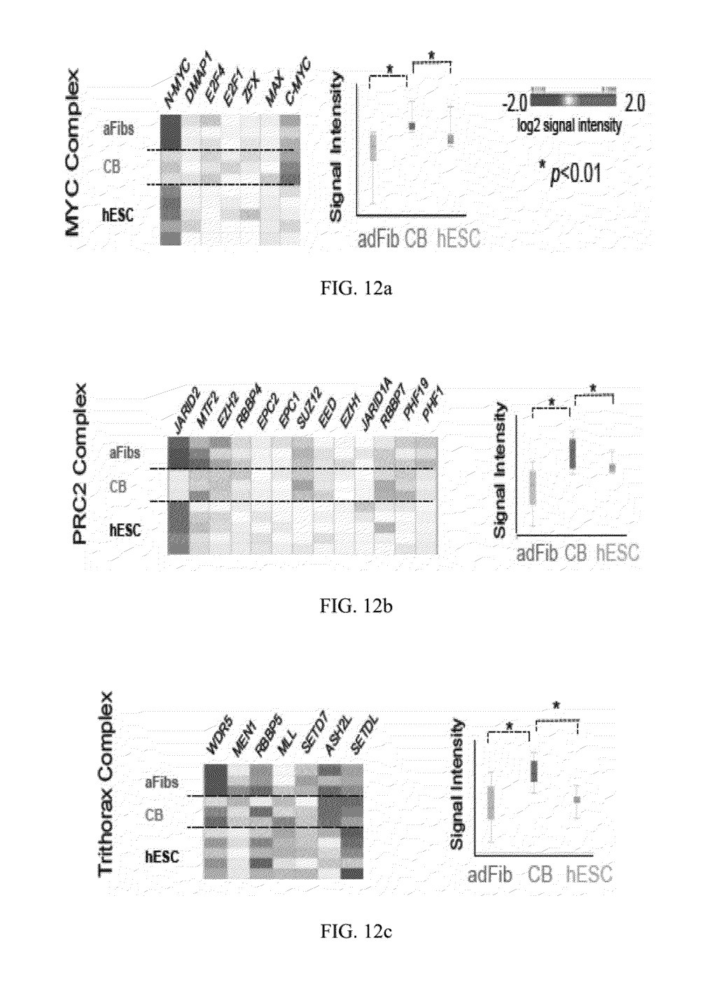

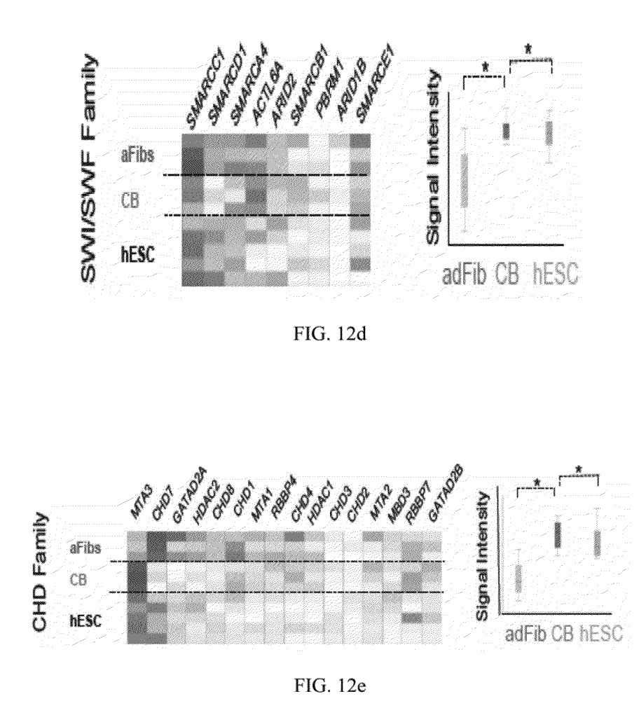

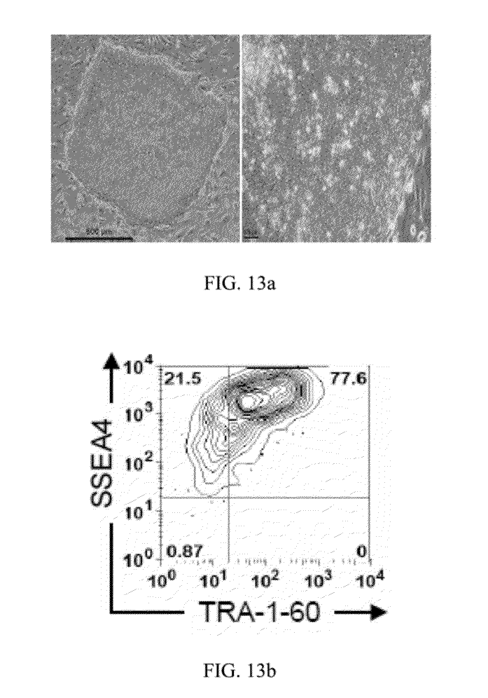

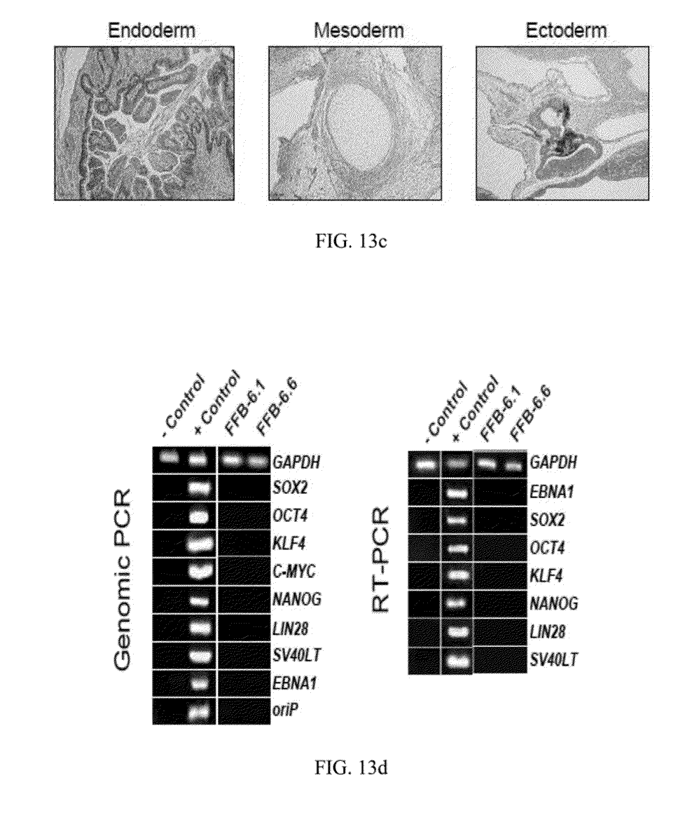

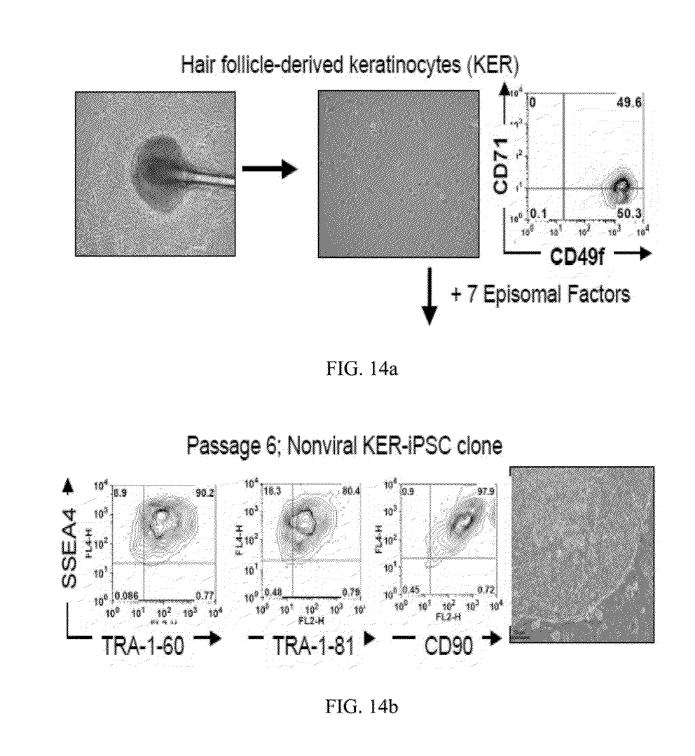

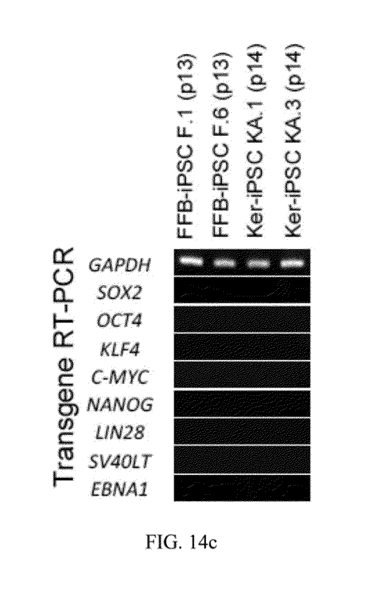

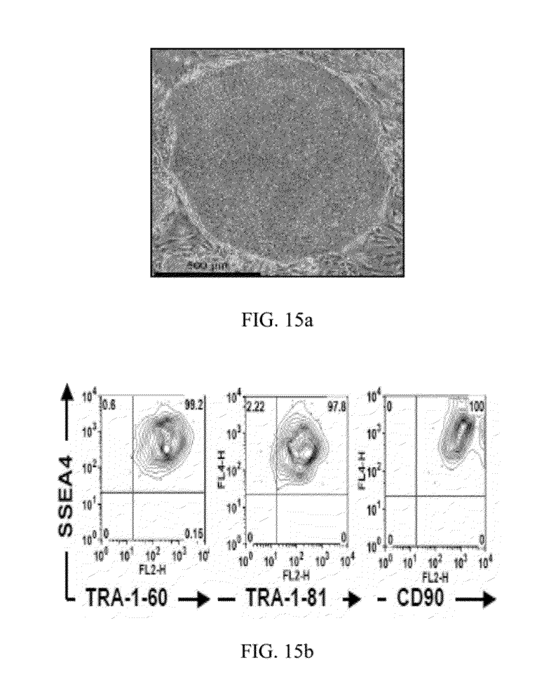

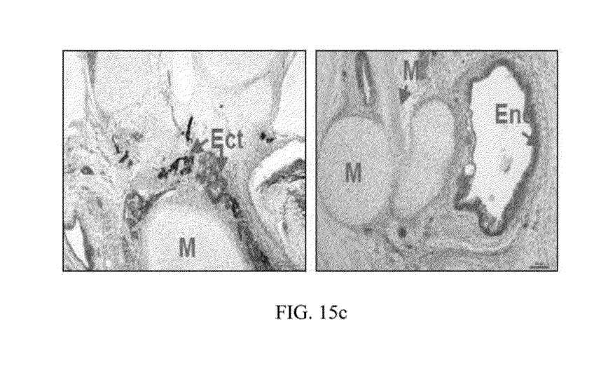

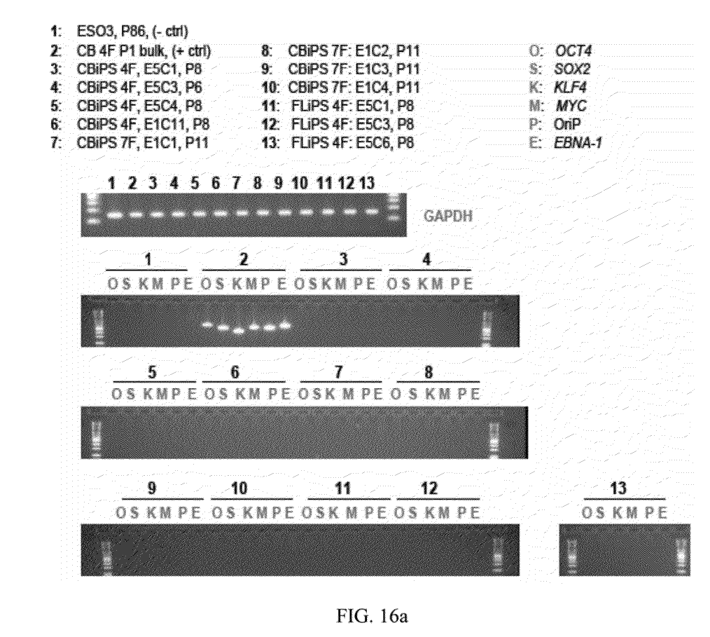

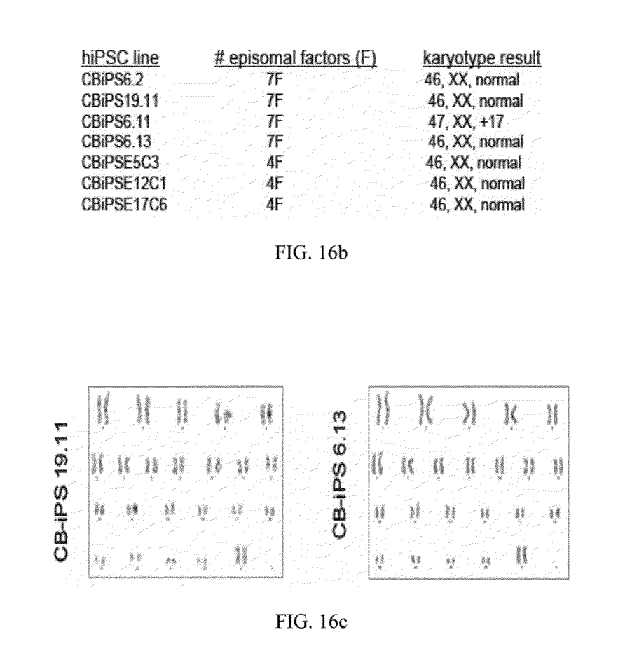

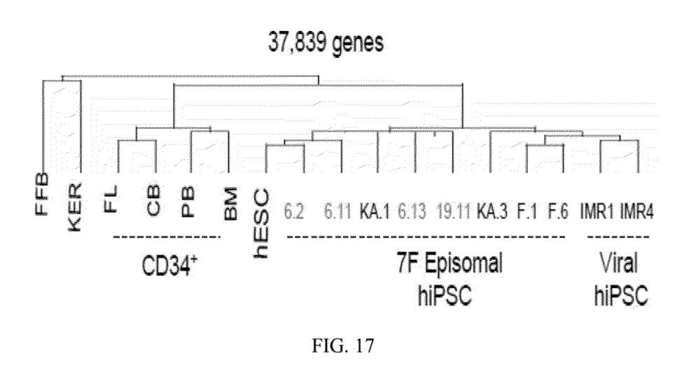

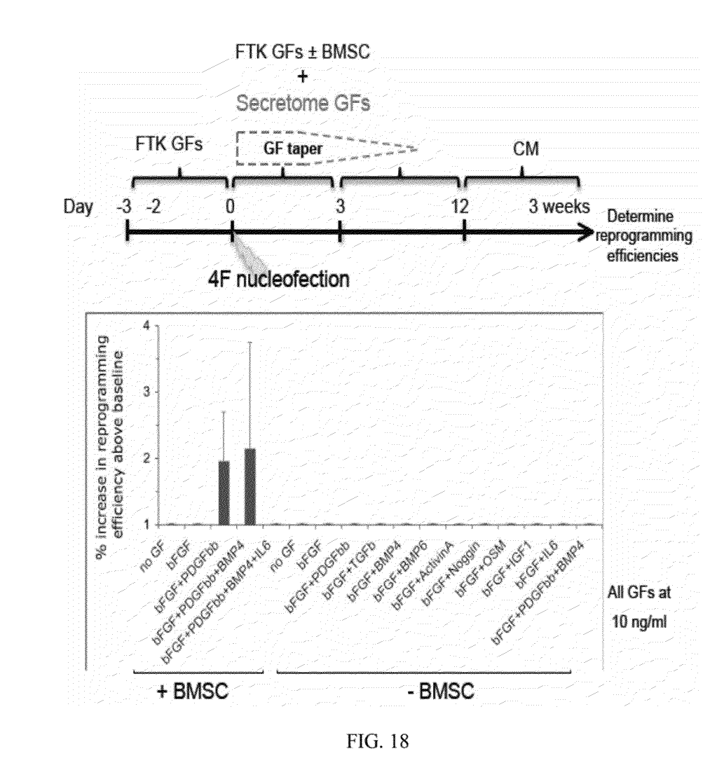

In FIG. 1b, brief co-culture of GF-activated CB cells with irradiated BMSC (+BMSC) did not increase the frequency of cells in G1 or S cell cycle phases at Day 3 (D3) of the reprogramming protocol (see FIG. 9) compared to GF alone (−BMSC). CB cells were nucleofected on Day 0 (D0) with 4F or 7F plasmids, or nucleofected with Amaxa buffer only (Mock). Cell cycle status of stromal-activated D3 CB, DO (pre-nucleofected) control CB cells, and adult fibroblasts (HDF1, HUF5 (FIBS); nucleofected with 7F) was determined on Day 3 of reprogramming protocols via FACS analysis of EdU incorporation and DNA content (7AAD), as described in the Examples section. Cells in G1 phase were defined as being EdU negative and 7AAD(2n), % cells in S phase were determined by gating on EdU positive 7AAD(2n+) populations. Data shown is the average of 2 experiments from individual batches of pooled CB donors run in triplicate.

FIG. 1c shows the episomal reprogramming efficiencies of various somatic targets with either four factors (4F; SOKM, on a single plasmid, pEP4 EO2S EM2K) or seven factors (7F; SOKMNLT, on three separate plasmids), and with (+) or without (−) BMSC priming were determined by AP staining (in triplicate) at 3 weeks following episomal nucleofections for CB progenitors (CB), fetal fibroblasts (FFB), adult fibroblasts (AdFib), and adult keratinocytes (Ker). Reprogramming efficiencies were also quantitated in parallel with live TRA-1-81 staining and gave similar results to AP staining (data not shown). Each condition presented was repeated at least two to five times, as indicated in panels, with significance (t test; *=p<0.05) where indicated. 4F SOKM reprogramming of CB progenitors was even more robust than with equimolar DNA quantities of the 7F three-plasmid system (albeit initially less rapid). CB-iPSC emerged rapidly with 4F from BMSC-primed CD34⁺ CB cells by 7-14 days at significantly higher frequencies (p<0.05) than without BMSC co-culture (4.3% average iPSC efficiency per input cells with 4F at 3 weeks (n=5, range: 3%-9.3% efficiencies). Notably, co-culture of AdFib with 3 days of irradiated BMSC did not improve the poor efficiency of non-integrated fibroblast-iPSC generation. ND; BMSC co-culture was not performed. With omission of the brief BMSC activation step, rarer (˜10-150-fold less) and slower-emerging CB-iPSC were produced with single-plasmid 4F nucleofections, but at frequencies that were still significantly greater than 7F reprogramming of fetal and adult fibroblasts, or adult keratinocytes.

FIG. 1d shows that completion of reprogramming was more rapid in bulk cultures of BMSC-primed CB progenitors. Emergence of surface pluripotency markers (SSEA4, Tra-1-81) at 3 weeks in bulk cultures of episomally-reprogrammed somatic cells briefly co-cultured with (+) or without (−) irradiated BMSC. Abbreviations: fetal fibroblasts (FFB), adult fibroblasts (AdFib), adult keratinocytes (Ker), GF-activated CB (CB). Somatic cells were nucleofected with 4F or 7F, and bulk cultures of reprogrammed cells were analyzed by FACS 3 weeks later. Shown are the averaged results of 2-5 experiments with averages, and significances designated at peak of bar graphs. By three weeks following nucleofection, CB cells nucleofected with 4F generated P₀ bulk populations and plated on MEF had already converted with near total efficiencies to 80-100% partially-reprogrammed SSEA4⁺ and 50-80% fully reprogrammed SSEA4+Tra-1-81⁺ NANOG⁺ CD34-iPSC.

FIG. 2 illustrates episomal 4F reprogramming of FACS-purified hematopoietic populations. The FIG. 2 schematic summarizes the strategy for determining the true reprogramming efficiency of hematopoietic progenitors via FACS purification of (FIG. 2a) lineage-committed and (FIG. 2b) episomal transgene-expressing myeloid populations. 4F reprogramming efficiencies of sorted CB populations were determined by AP⁺ staining of ESL colonies. Experimental details are provided in the Examples section. Post sort analysis of FACS-purified Day 0 CD34⁺CD38⁺ CB fractions verified that >95% of this populations consisted of CD33⁺CD13⁺CD45⁺ myeloid cells (FIG. 10). Reprogramming efficiency (AP staining) and reprogramming completion (bulk SSEA4⁺TRA-1-81⁺ and NANOG⁺ staining) assays were conducted 4 weeks following 4F CB nucleofections with a single plasmid (pEP4 EO2S EM2K) expressing the four Yamanaka factors on P₀ MEF cultures.

FIG. 2a presents a representative AP staining (plates done in triplicate, with indicated number of ESL colonies emerging per the number of single sorted CB cells plated on MEF, i.e., unsorted CB vs. CD34⁺CD38^{lo} vs. CD34⁺CD38⁺ fractions), with the averaged results of two independent experiments indicated. In lower panels are shown representative FACS staining of surface (TRA-1-81) and intracellular (NANOG) pluripotency markers of bulk reprogrammed CB cultures at 4 weeks of P₀ MEF/CM cultures.

As shown in FIG. 2b, to determine the true reprogramming efficiency of myeloid populations that had been successfully nucleofected, CB cells were co-nucleofected on day 0 with both the 4F pEP4 EO2S EM2K, and a pCEP4-GFP episomal construct. Episomal SOKM transgene expressing-only populations were purified by GFP expression prior to plating on Day 3 MEF and determining reprogramming efficiency. CB cells were stained on Day 3 with CD34-PE and FACS-purified into episome-expressing (GFP⁺CD34⁺, GFP⁺CD34), and non episome-expressing (GFP)+/−BMSC-primed populations prior to MEF plating for reprogramming efficiency determinations. Results of AP stains plates shown are representative of independent sorting experiments using pooled donor CB samples for each 4F nucleofection, with the averaged results of two independent experiments indicated below.

FIG. 3 shows endogenous expression of pluripotency factors in parental donor populations. In FIG. 3a, endogenous expressions of pluripotency-associated factors were determined by qRT-PCR analysis on Day 0 of the reprogramming protocol of GF-activated (FTK) donor cell populations (CD34⁺ fetal liver; FL), CD34⁺ cord blood (CB), GCSF-mobilized peripheral CD34⁺ blood (mPB), adult CD34⁺ bone marrow (BM), adult keratinocytes (KER), and fetal fibroblasts (FFB). FIG. 3a shows the fold change normalized expression levels of each factor relative to expression in control H9 hESC calculated by the 2^{-ΔΔCT} method. Primer sequences are presented in the Examples section. In FIG. 3b, stem-progenitor)(CD34⁺CD38^{lo}) and lineage-committed (CD34⁺CD38⁺) populations were FACS-purified from Day-2 CB cells, and similarly evaluated for expression of endogenous pluripotency factor transcripts by qRT-PCR.

FIG. 4 demonstrates that partially-reprogrammed stem cell modules in GF-activated hematopoietic progenitors with rapid reconfiguration to ESC-like patterns. FIG. 4a presents Illumina microarray expressions of pluripotency-associated gene modules (MYC, PRC1, PRC2, Core; FIG. 19/Table S1) in hESC, bulk day 23 CB-iPSC cultures, somatic fibroblasts, and CB donors with (+) and without (−) 4F episomal nucleofections, and with (+) and without (−) BMSC-priming. Adult fibroblasts (F), Day −3 unstimulated CB cells (D-3 CB), Day 0 FTK GF-stimulated CB cells (D0+/−BMSC). Day 3 (D3) CB for gene microarray samples were FACS-purified from irradiated BMSC with CD45 surface staining. Viable bulk Day 23 early CB-iPSC culture (D23 iPSC) samples for microarrays were also FACS-purified from nonviable irradiated MEF. These early populations were already composed of >50-60% populations with fully-reprogrammed TRA-1-81⁺NANOG⁺ phenotypes. Undifferentiated H9 hESC samples served as control (hESC). Module expressions represent log 2 mean-subtracted normalized values of signal intensities from averaged, independent biological replicate microarray samples (n=3 per condition). In mean normalization, each gene's mean log 2 signal value is determined for all the cell types, and then subtracted from each cell type's signal intensity value for that gene. Although they possessed a transcriptionally-inactive Core module, GF-activated CB progenitors expressed active ESC and MYC modules, and inactive PRC1, and PRC2 modules at mean expression levels that were already comparable to levels in hESC. The annotation and references of all genes in each module is provided in FIG. 19/TABLE S1.

FIG. 4b shows the partially-reprogrammed ESC module in CB progenitors. The legend for samples is the same as FIG. 4a above. FIG. 4b shows unsupervised hierarchical clustering heat maps of expression and FIG. 4c provides box plots of log 2 mean-normalized values of the ESC module gene signal intensities in somatic target populations, hESC, and reprogrammed cell lines. The heat map's color scale was chosen to emphasize subtle mid-range change. The resulting values emphasize relative expression across cell types rather than relative absolute expression across genes. This box and whisker plots (right panels) depict the log 2 mean-subtracted normalized values of signal intensities of genes comprising the module set for each cell type indicated from Illumina array data. The top and bottom of a box (FIG. 4c) mark the 75^{th} and 25^{th} percentile log 2 signal values, respectively, while the bar at the middle denotes the median. The whiskers above and below each box mark the upper 90^{th} and lower 10^{th} percentiles. Paired tests with significance p<0.05 (*) or without significance (NS; p>0.05) with values of control hESC are indicated.

As shown in FIG. 5, GF-activated hematopoietic progenitors expressed an active ESC-like OCT4 interactome network and chromatin remodeling factors known to augment iPSC generation. FIG. 5a: unsupervised hierarchical clustering heat map of expression of the OCT4 interactome module (FIG. 19/TABLE S1) in fibroblasts (F), CB progenitors at various stages of the reprogramming protocol (D-3, D0, D3+/−BMSC), day 23 bulk early CB-iPSC cultures (D23 iPSC), and hESC(H9) controls; gene arrays samples (n=3 per condition) are the same as defined above. Box and whisker plots of same samples of the log 2 mean-subtracted normalized values of signal intensities of gene module sets for (FIG. 5b) a subset of the OCT4 interactome consisting of ESC-regulating epigenetic modulators (see FIG. 19/TABLE S1 for list). FIG. 5c: the MYC transcription factor complex (N-MYC, C-MYC, E2F4, E2F1, ZFX, MAX). FIG. 5d: the PRC2 repressive complex (JARID2, MTF2, EZH2, RBBP4, EPC2, EPC1, SUZ12, EED, EZH1, JARIDIA, RBBP7, PHF19, PHF1).

As shown in FIG. 6, reprogramming efficiency in developmentally progressed GF-activated hematopoietic progenitors correlates directly to expression levels of ESC-like circuits.

In FIG. 6a, 7F (SOKMNLT) reprogramming efficiencies of developmentally progressing GF-primed Day 0 hematopoietic progenitors were simultaneously determined in parallel 3 weeks following 7F nucelofections with two independent methods of 1) AP⁺ staining (top panels) and 2) live TRA-1-81 staining (bottom panels; shown with merged brightfield (BF) images) of ESL colonies, as described in the Examples section. Note that live TRA-1-81 staining, which indicates conversion to a completed reprogrammed state, emerged from ESL colonies with slower kinetics and in a more heterogeneous pattern than AP-positivity of ESL colonies. FIG. 6b: 7F reprogramming efficiencies. BMSC-primed Day 0 hematopoietic progenitors (FL, CB, inn, BM), keratinocytes (KER), or fetal fibroblasts (FFB) populations were reprogrammed with non-integrated 7F episomes as described in text. Log 2 mean-normalized microarray expressions (signal intensities) in somatic target populations (from pooled Day 0 GF-primed CD34⁺ donors; n=3-4 per sample) and h9 hESC of (FIG. 6c) MYC complex genes, (FIG. 6d) MYC-regulated ESC module (FIG. 6e) MYC module, (FIG. 6f) the OCT4 interactome, and (FIG. 6g) PRC2 complex genes (see FIG. 19/TABLE S1).

In FIG. 7, episomal CB reprogramming was accelerated by paracrine and contact-dependent signals provided by the stromal niche. FIG. 7 shows the kinetics of pluripotency marker emergence of BMSC-primed 4F reprogrammed CB progenitors. FIG. 7a: SSEA4⁺ and FIG. 7b: SSEA4⁺TRA-1-60⁺ expressions. FIG. 7c: enhancement of 4F CB reprogramming with BMSC priming was due to stromal signals that were partially cell contact-dependent, and partially soluble factor-mediated. GF-activated CB cells were cultured as described in FIG. 9 from Day 0 until Day 3 without BMSC co-culture (−BMSC), with BMSC co-culture (+BMSC), or with BMSC co-culture but physically separated from CB cells with a Transwell insert that prevented cell-cell contact between BMSC and CB cells, but allowed diffusion of soluble stromal factors (+BMSC(T)). Shown is the fold-increase of reprogramming efficiency (enumerated AP+ ESC-like colonies) from two averaged 4F-reprogramming experiments from baseline efficiencies (−BMSC conditions). Reprogramming efficiency was determined at 3 weeks post-nucleofection with 4F, determined by AP staining of ESC-like colonies (as described in the Examples section).

FIG. 7d: GSEA analysis of pathways activated in CB cells by stromal signals. The GSEA algorithm was used to identify curated Reactome pathways over-represented among genes with significant (p<0.05) differential expression between the following independently paired expression array sets (n=3 experiments per condition): 1) Day 0 (D0) 4F-nucleofected CB samples vs. day 3 (D3)+BMSC-primed CB samples, and 2) D0 4F-nucleofected CB samples vs. D3 without (−) BMSC primed CB samples. Table S3 summarizes the MSigDB v. 3.0 Reactome gene set categories that were enriched with FDR<=0.05 in these two paired gene set computations. Shown is the summary from Table S3 of GSEA enrichment plots of the four pathways in D3 BMSC-primed CB samples that were positively enriched (red) or under-expressed (blue) relative to D0 CB samples and D3-BMSC samples.

FIG. 8: Generation of non-integrated episomal CB-iPSC from BMSC-primed CB progenitors. The model for efficient BMSC-primed CB progenitor reprogramming: GF-activated hematopoietic progenitors already express pluripotency-associated transcriptional modules de novo at ESC-like levels. These networks are subsequently facilitated to a stable pluripotent state via a synergy between stromal signals and transient ectopic expression of the Yamanaka factors. MYC (MYC-regulated MYC and ESC gene modules; OCT4-1 (OCT4 interactome module); PcG (PRC1, PRC2 gene modules); Core (SOX2-OCT4-NANOG-regulated genes module).

FIG. 9 is a summary of the experimental design for determining comparative episomal reprogramming efficiencies of human somatic target cells. Reprogramming efficiencies of GF-activated and +/−BMSC-primed CB progenitors (FIG. 9a) or fetal and adult fibroblast (FFB; AdFib) and adult keratinocyte (Ker) populations (FIG. 9b) were determined on MEF cultures (plated on day +3) following plasmid nucleofections on Day 0 with four (4F) or seven (7F) episomal transgenes. Day 0 nucleofected CB cells were briefly co-cultured with (or without) irradiated adult BMSC stromal layers and continued hematopoietic GFs (Flt3L, TPO, Kit ligand-SCF (FTK)) from Day 0 to Day +3.

Reprogramming efficiencies of emerging CB-iPSC colonies were determined on initial (P0) MEF cultures at day 3-5 weeks post nucleofections. Medium was replaced daily with MEF-conditioned medium (CM) supplemented with 40 ng/ml bFGF after 12 days on MEF. Reprogramming efficiencies for somatic targets were determined via two independent methods in averaged triplicate-quadruplicate cultures for each experiment by counting the number of iPSC colonies emerging per single cells plated on replicate P0 MEF cultures at day 21 that had ESL morphology (as defined by compact embryonic stem cell characteristics with large nuclei and nucleoli and high alkaline phosphatase activity (AP+; AlkPhoshi). Alternatively, ESL colonies that were positive for live Tra-1-81 surface staining were enumerated in replicate cultures. ESL/AP+/Tra-1-81+ colonies emerged from nucleofected CB as early as 7-21 days post-nucleofection. Both efficiency assays gave comparable results and AP+ assays are described herein. Additionally, because a large majority of BMSC-primed CB cells converted to ESL-like colonies, in some experiments, the completion of reprogramming in whole populations of actively-reprogramming cells was estimated via FACS expression of intracellular NANOG, and surface TRA-1-81 and SSEA4 of whole, bulk cultures.

Unlike 4F or 7F-nucleofected CB, 7F-nucleofected keratinocytes and adult or fetal fibroblast cells never produced ESL colonies on initial P0 MEF and CM cultures at 3-5 weeks. Episomal fibroblast-iPSC, and keratinocyte-iPSC colonies emerged rarely for these donor types. Thus, bulk P0 cultures for fibroblasts and keratinocyte reprogramming experiments were passaged after 4 weeks with 1 mg mL-1 of collagenase IV onto fresh irradiated MEF layers (P1) at a ratio of 1:1-1:6) for further expansion of slowly reprogramming precursors. Estimated efficiencies for fibroblast-iPSC and keratinocyte-iPSC were determined on these secondary P1 MEF cultures several weeks later.

FIG. 10: Brief co-culture of growth factor-activated day 0 CB cells with BMSC preserved multipotent hematopoietic progenitor frequencies. Brief co-culture of GF-activated (from Day −3 to Day 3) CB cells with irradiated BMSC for an additional 3 days (from Day 0 to Day 3 of reprogramming protocol; see FIG. 9) increased the frequency of multipotent hematopoietic CD34+CD45+ progenitors (FIG. 10a) and erythro-myeloid GEMM-CFU (FIG. 10b) (and to a lesser extent in mobilized CD34+ peripheral blood progenitors (mPB)). CFU colony assays of GF-activated Day 0 CB cells were conducted in semi-solid methylcellulose as previously described. FIG. 10c: by Day 3, both CD34+ and CD34-GF-activated CD45+CB progenitors maintained primarily a myeloid CD33+ and CD13+(not shown) phenotype.

FIG. 11 shows that nucleofection of large episomal plasmids into Day 0 CB cells is inefficient. Gene transfer efficiency of Day 0 CB, adult human fibroblasts, or 293T embryonic kidney carcinoma cells was determined by GFP reporter expression with either a 3 kb CMV-GFP plasmid (pMAXCMV-GFP; AMAXA kit) or a ˜15 kb EBNA-based GFP episome (pCEP4-EF1-GFP) of similar size, and with the same promoter and vector backbone as our reprogramming plasmids. FIG. 11a: results of averaged experiments for 48 hr GFP expression of Fibs or CB cells nucleofected on Day 0 with 6 μg plasmids per 500,000 cells. Time courses of GFP expression following 293T transfections (Lipofectamine 2000) (FIG. 11b), or BMSC-primed CB nucleofections of each plasmid (FIG. 11c). These experiments revealed that our large pCEP4 EBNA-based episomes were excellent expression vectors via transfection, but possessed limiting nucleofection gene transfer efficiency, likely due to their large sizes. A second pulse of plasmid (FIG. 11c, 2nd nucl) was nucleofected on day 3 in some experiments, but did not dramatically improve the low gene transfer efficiency of the original pulse (1st nucl).

FIG. 12: Endogenous expression of chromatin remodeling factors in parental donor cells. Relative expressions of genes in chromatin remodeling factor families. genes MYC complex (FIG. 12a), PRC2 complex genes (FIG. 12b), Trithorax complex genes (FIG. 12c), SWI/SWF family genes (FIG. 12d), Chromodomain (CHD) family genes in parental donor samples (FIG. 12e) (Day 0 GF-activated CB; pooled CB donors; n=3 samples), adult fibs (aFibs; n=3), hESC (n=5)). Heat maps and boxplots of individual samples were generated from log 2 mean normalized subtracted values of Illumina microarray signal intensities (y-axes) with statistical approaches, as described in the Examples section.

FIG. 13: Generation of pluripotent non-integrated 7F episomal hiPSC from fibroblasts with EBNA 1-based episomal plasmids. Twenty-two week-old lung fetal fibroblasts carrying the homozygous sickle cell disease mutation were obtained from the Coriell Cell Repository (GM02340), and used to generate non-viral human fetal fibroblast-derived SSEA4+Tra-60+hiPSC (FIGS. 13a and 13b) with seven episomal factors, as described herein, that demonstrated differentiation to all three germ layers in NOG teratoma assays FIG. 13c). Shown are H&E stains of teratoma sections from SCD-hiPSC demonstrating elements of ectoderm (neural rosettes, retinal pigmented epithelium, endoderm (glandular epithelium), and mesoderm (bone, muscle). Shown also are genomic PCR and RTPCR assays (FIG. 13d) confirming the lack of integration and expression of transgenic episomal constructs. For details regarding these transgene-specific PCRs, see Burridge et al., 6(4) PLoS ONE e18293. doi:10.1371/journal.pone.0018293 (2011).

FIG. 14: Generation of pluripotent non-integrated 7-factor episomal hiPSC from normal adult hair follicle keratinocytes. Keratinocyte lineage cells were confirmed by CD49f(alpha-integrin)-positive, CD71-low cells, after expansion from a single plucked hair (FIG. 14a; left panel) of a normal adult donor, using methods as described previously. About 2×106 cells were nucleofected with “Combo 6”, re-suspended in fresh culture medium, and then transferred onto gelatinized PMEF plates. After 48-72 hours, media was replaced with hESC medium or CM for three weeks (P0), followed by replating onto fresh PMEF (P1). FIG. 14a: colonies with hESC-like (ESL) morphology, and expressing pluripotency markers (e.g., SSEA4, Tra-1-60/81, CD90, OCT4, NANOG, SOX2), emerged with rare efficiencies (see FIGS. 1-2) 1-2 weeks following P1 culture of nucleofected cells. Non-viral iPSC clones derived from keratinocytes. KERiPSC were further subcloned, and confirmed for lack of integrated episomal sequences by genomic PCR, and RTPCR (FIG. 14c) of pluripotency transgenes, expanded for frozen stocks, and confirmed for pluripotency by tri-lineage cystic teratoma formation assay (not shown).

FIG. 15: Generation of non-integrated 4F and 7F episomal CB-iPSC lines. Representative colony morphology (FIG. 15a) and FACS staining (FIG. 15b) of SSEA4, TRA-1-81, and CD90 surface pluripotency markers from CB-iPSC lines generated as described in the Examples section, with four (4F) or seven (7F) episomal reprogramming factors. Full characterizations, including Southern Blots, Genomic PCR for validations of vector and transgene-free status of the CB-iPSC lines that were evaluated by expression microarrays in FIG. 16 (e.g., clones 6.2, 6.11, 6.13, and 19.11) were previously reported (Burridge et al., 2011). FIG. 15c: H&E stains of cystic teratomas obtained from a representative CB-iPSC line 6-8 weeks following injection into NOD/SCID mice illustrate well-differentiated cell lineages of all three germ layers, including regions containing neural rosettes, pigmented retinal epithelium, glandular epithelium, fetal intestinal structures, cartilage, striated muscle, and hyalinized bone. Ectodermal structures (Ect): neural rosettes (left); retinal pigmented epithelium (right); Endodermal structures (End): glandular epithelium (left); developing gut loop (right); Mesodermal structures (M): cartilage (left), bone/muscle (right). All CB-iPSC lines described herein formed similar tri-lineage cystic teratomas. Analysis of histological sections also demonstrated that these teratomas were completely devoid of foci of malignant transformation. Scale bars=100 μM.

FIG. 16: Episomal integration and karyotypes of non-integrated 4F and 7F episomal hiPSC derived from CD34+CB and FL progenitors. FIGS. 16a and 16b: 4F and 7F CB-iPSC and FL-iPSC were assayed by transgene-specific genomic PCR at indicated passages exactly as previously described (Burridge et al, 2011) for episomal sequences. Bulk P1 4F CB-iPSC cultures serve as a positive control. FIG. 16c: G-band karyotyping. Experimental details are described in the Example section.

FIG. 17: Genome-wide expression studies of non-integrated hiPSC lines revealed that stromal-primed low passage CB-iPSC lines possessed transcriptional signatures that were highly akin to hESC at low passage. To examine the quality of non-integrated reprogramming achieved, low passage hiPSC clones were derived from fetal fibroblasts (FIG. 13), keratinocytes (FIG. 14), as well as stromal-primed CB donors (FIG. 15-16). Non-integrated hiPSC were generated with the same 7F episomal constructs, and global gene expressions were compared. All non-integrated hiPSC lines were confirmed to be free of transgene and vector sequences by Southern blotting, genomic PCR, and RT-PCR at early passage (p9-12), as previously described (Burridge et al, 2011; and FIGS. 13-16). Levels of pluripotency markers SSEA4, TRA-1-60, TRA-1-81, OCT4, and NANOG proteins for all hiPSC assayed were found comparable to control hESC. All non-integrated iPSC lines were also tested for their ability to form, well-differentiated tri-lineage cystic teratomas in NOG-SCID mice demonstrating their bona fide pluripotency.

The expression signatures of these non-integrated hiPSC clones was determined with Illumina microarrays, and also included previously described lentiviral hiPSC lines IMR90-1 and IMR90-2 and H9 hESC as controls. An unsupervised hierarchical clustering of global expression (37,839 genes) from all starting populations and cell lines was computed. Global gene expression samples of episomal lines was evaluated at the earliest passage possible (P₁₁₋₁₄). H9 hESC(P₅₁), episomal CB-iPSC5 clones 6.2, 6.11, 6.13, (P₁₄), 19.11, (P₁₁), non-viral keratinocyte-iPSC clones: KA.1, KA.3 (P₁₃); episomal fetal fibroblast-iPSC: F.1, F.6 (P₁₄); viral fibroblast-iPSC clones: IMR1 (P₆₆), IMR4 (P₆₄). This dendrogram represents the unsupervised hierarchical clustering of signal values from all 37,839 genes represented on the Illumina microarray for all cell types examined. Low passage (P₁₁₋₁₄) non-integrated CB-iPSC samples (n=4) had global expression profiles that highly correlated to hESC (Pearson coefficients R²=0.98). Fibroblast-iPSC and keratinocyte-iPSC had Pearson coefficients of R²=0.96 relative to hESC. Collectively, these studies revealed that 1) CD34+ progenitor populations (FL, CB, BM, mPB) were transcriptionally more akin to pluripotent stem cells as a group, 2) low passage (P₁₁₋₁₄) non-integrated CB-iPSC samples (n=4) had global expression profiles that more faithfully correlated (Pearson coefficients R²=0.98) with those of control hESC, and 3) stromal-primed reprogramming could generate high quality CB-iPSC that resembled hESC at low passages.

FIG. 18: The effect of stem cell growth factors identified in CB-BMSC secretome studies on further augmentation of 4F CB myeloid progenitor reprogramming. To probe the identities of soluble factors generated by CB-BMSC interactions that may augment reprogramming efficiency, the top 10 stem cell growth factors identified from CB-BMSC secretome studies were tested for their ability to further enhance stromal-primed (+BMSC) or unprimed (−BMSC) CB reprogramming (Table S2, data not shown). Combinations of recombinant GFs (R&D Systems), as indicated above, were added at 10 ng/ml from Day 0 to Day 3 of the reprogramming protocol (see FIG. 18 schematic above and FIG. 9), and then again as a single bolus from Day 3 onwards following passage of single cells onto MEF (along with baseline Flt3L, TPO, and SCF (FTK), as described in the Examples section). Emerging ESC-like iPSC colonies were enumerated as before by AP+ staining 3 weeks later in triplicate cultures. Shown is the % increase of reprogramming efficiency above the baseline of +BMSC conditions. All culture conditions included 40 ng/ml bFGF during MEF cultures following Day 3. However, where ‘bFGF’ is indicated above, bFGF was also included earlier, starting on Day 0 at 40 ng/ml during the +/−BMSC priming step.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

As described herein, the present invention identifies important synergies between hematopoietic regulatory circuits activated by growth factors (GFs), and extrinsic niche factors that efficiently direct the induction of myeloid cells to high-quality human induced pluripotent stem cells (hiPSC). Efficient pluripotency induction correlated not to increased proliferation or endogenous myeloid expression of either individual Core factors (e.g. SOX2, OCT4, NANOG; SON) or Core-regulated circuits, but to expression of ESC-like levels of MYC and OCT4-associated circuits, and inactivated ESC-like Polycomb group (PcG)-regulated networks. These circuits were all poised in partially-reprogrammed states prior to ectopic episomal factor expression (FIG. 8). The reprogramming efficiency of bone marrow stromal cell (BMSC)-primed progenitors, which was 3-10% in unfractionated cord blood (CB) cells, and 50-65% in purified episome-expressing myeloid cells was 3-4 logs greater than the efficiency of deriving episomal hiPSC from fibroblasts or hair follicle-derived keratinocytes. The green fluorescent protein (GFP) purification experiments demonstrated that CB-iPSC were emerging not from a minority population, but from the majority of successfully-nucleofected myeloid cells. The application of methods with higher gene transfer efficiencies for expressing reprogramming factors (e.g. via synthetic mRNAs or microRNAs) may allow further optimization of this hematopoietic reprogramming system. More importantly, this experimental system opens new avenues of molecular, epigenetic and proteomic investigation for elucidating novel micro-environmental cellular factors that drive rapid and efficient reprogramming in synchronized populations of donor cells in more defined conditions.

The term “reprogramming,” as used herein, refers to a process where cells of a differentiated state are converted into cells of a de-differentiated state. Reprogrammed cells can be pluripotent or multipotent cells.

The term “pluripotent cells” or “pluripotent stem cells” as used herein, refers to cells of an undifferentiated or a de-differentiated state and can differentiate into various cell types. Pluripotent cells express pluripotent cell-specific markers, and have a cell morphology characteristic of undifferentiated cells (e.g., compact colony, high nucleus to cytoplasm ratio, and/or prominent nucleolus). Typically, pluripotent cells can be induced to differentiate into all three germ layers (e.g., endoderm, mesoderm and ectoderm).

The terms “pluripotency factors”, “pluripotency induction factors” and “defined factors” refer to factors/proteins/transcription factors and the like that are associated with the pluripotency of a cell. Similarly, the term “pluripotency gene” refers to a gene that is associated with the pluripotency of a cell. Typically, a pluripotency factor is expressed only in pluripotent stem cells and is crucial for the functional identity of pluripotent stem cells.

Specific examples of pluripotency factors include, but are not limited to, glycine N-methyltransferase, Nanog, GABRB3, LEFTB, NR6A1, PODXL, PTEN, REX-1 (also known as ZFP42), Integrin α6, ROX1, LIF-R, TDGF1 (CRIPTO), SALL4, leukocyte cell derived chemotaxin 1 (LECTI), BUBI, FOXD3, NR5A2, TERT, LIFR, SFRP2, TFCP2L1, LIN28, XIST and simian virus 40 large-T antigen (SV40LT). The term also includes the “Yamanaka factors”, namely, sex-determining region Y HMG box 2 (SOX2), octamer binding transcription factor 4 (OCT4), Kruppel-like factor 4 (KLF4), v-myc myelocytomatosis viral oncogene homolog (c-Myc or MYC).

As used herein, the term “mesenchymal stromal cells” (MSCs), or “mesenchymal stem cells”, refers to multipotent cells naturally found inter alia in bone marrow, blood, dermis and periosteum that are capable of differentiating into more than one specific type of mesenchymal or connective tissue (i.e., the tissues of the body that support the specialized elements; e.g., adipose, osseous, stroma, cartilaginous, elastic and fibrous connective tissues) depending upon various influences from bioactive factors, such as cytokines. Moreover, MSCs of the present invention adhere to plastic when maintained in standard culture conditions; express one or more of CD 105, CD73 or CD90; and lack expression of one or more of CD45, CD34, CD 14, CD1Ib, CD79alpha, CD19 or HLA-DR.

As used herein, “isolated” signifies that the cells are placed into conditions other than their natural environment; however, the term “isolated” does not preclude the later use of these cells thereafter in combinations or mixtures with other cells.

Any appropriate method can be used to introduce a nucleic acid (e.g., nucleic acid encoding pluripotency factors) into a cell. For example, nucleic acid encoding the Yamanaka factors (e.g., SOX2, OCT4, KLF4 and MYC) designed to induce pluripotent stem cells from other cells (e.g., non-embryonic stem cells) can be transferred to the cells using liposomes or other non-viral methods such as electroporation, microinjection, nucleofection, transposons, phage integrases, or calcium phosphate precipitation, that are capable of delivering nucleic acids to cells.

The exogenous nucleic acid that is delivered typically is part of a vector. Standard molecular biology techniques suitable for use in the subject invention for the construction of expression vectors are known to one of ordinary skill in the art and can be found in Sambrook et ah, “Molecular cloning: a laboratory manual,” (3rd ed. Cold Spring harbor Press, Cold Spring Harbor, N.Y. 2001), which is incorporated by reference in its entirety.

In particular vector embodiments, a regulatory element such as a promoter is operably linked to the nucleic acid of interest (i.e., a pluripotency gene). The promoter can be constitutive or inducible. Non-limiting examples of constitutive promoters include cytomegalovirus (CMV) promoter and the Rous sarcoma virus promoter. As used herein, “inducible” refers to both up-regulation and down regulation. An inducible promoter is a promoter that is capable of directly or indirectly activating transcription of one or more DNA sequences or genes in response to an inducer. In the absence of an inducer, the DNA sequences or genes will not be transcribed. The inducer can be a chemical agent such as a protein, metabolite, growth regulator, phenolic compound, or a physiological stress imposed directly by, for example heat, or indirectly through the action of a pathogen or disease agent such as a virus.

Additional regulatory elements that may be useful in vectors include, but are not limited to, polyadenylation sequences, translation control sequences (e.g., an internal ribosome entry segment, IRES), enhancers, or introns. Such elements may not be necessary, although they can increase expression by affecting transcription, stability of the mRNA, translational efficiency, or the like. Such elements can be included in a nucleic acid construct, as desired, to obtain optimal expression of the nucleic acids in the cells. Sufficient expression, however, can sometimes be obtained without such additional elements.

Vectors also can include other elements. For example, a vector can include a nucleic acid that encodes a signal peptide such that the encoded polypeptide is directed to a particular cellular location (e.g., the cell surface) or a nucleic acid that encodes a selectable marker. Non-limiting examples of selectable markers include puromycin, adenosine deaminase (ADA), aminoglycoside phosphotransferase (neo, G418, APH), dihydrofolate reductase (DHFR), hygromycin-B-phosphtransferase, thymidine kinase (TK), and xanthin-guanine phosphoribosyltransferase (XGPRT). Such markers are useful for selecting stable transformants in culture.

Any appropriate non-viral vectors can be used to introduce pluripotency factors, such as Oct3/4, Klf4, Sox2, and c-Myc. Examples of non-viral vectors include, without limitation, vectors based on plasmid DNA or RNA, retroelement, transposon, and episomal vectors. In one embodiment, vectors are delivered to cells via nucleofection, a type of electroporation. See the Nucleofactor technology from Lonza Cologne GmbH (Cologne, Germany). See also, Aluigi et al., 24(2) STEM CELLS 454-61 (2006); Pascal et al., 142(1) J. NEUROSCI. METHODS 137-43 (2005).

Non-viral vectors can also be delivered to cells via liposomes, which are artificial membrane vesicles. The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations. Transduction efficiency of liposomes can be increased by using dioleoylphosphatidylethanolamine during transduction. High efficiency liposomes are commercially available. See, for example, SuperFect® from Qiagen (Valencia, Calif.).

In one embodiment, the non-viral vector is an episomal vector. The episomal vector can include one or more pluripotency genes operatively linked to at least one regulatory sequence for expressing the factors. The episomal vectors of the invention can also include components allowing the vector to self-replicate in cells. For example, the Epstein Barr oriP/Nuclear Antigen-1 (EBNA-1) combination can support vector self-replication in mammalian cells, particularly primate cells. The EBNA1 trans element and OriP cis element derived from the EBV genome enables a simple plasmid to replicate and sustain as an episome in proliferating human cells. It can also persist episomally in human ESCs with little effect on their self-renewal and pluripotency. Episomal EBNA1/OriP plasmids delivered to human ESCs are lost gradually in the absence of any selection, likely due to epigenetic modification (such as DNA methylation) of the plasmid which leads to loss of EBNA1 expression and/or OriP functions.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Materials and Methods

Reprogramming Efficiency Determinations.

The experimental design for determining comparative reprogramming efficiencies in CB-iPSC, fibroblast-iPSC and keratinocyte-iPSC is summarized in FIG. 9. The efficiency of CB-iPSC generation from single unfractionated or FACS-purified CB populations was determined directly on MEF cultures following 6 days of GF stimulation (which included 3 days of +/−BMSC priming). The experimental details for episomal reprogramming of FACS-purified CD34⁺CD38^{hi} and CD34+CD38^{low} hematopoietic populations are provided further below. A schematic that summarizes the reprogramming strategy of FACS-purified hematopoietic populations, including the enrichment of lineage-committed myeloid progenitors that co-expressed reprogramming episomes and a GFP reporter is outlined in FIG. 2.

Reprogramming efficiencies were determined 3-5 weeks following episomal nucleofections on the original (P₀) MEF cultures (without additional subsequent MEF passages) via two independent methods. The number of colonies that emerged (per single input cells plated on day 3) possessing well-defined embryonic stem cell-like (ESL) borders, compact morphology, large nuclei, and rapid, strong high alkaline phosphatase (AP^{hi}) staining (Sigma-Aldrich, St. Louis, Mo.) were enumerated. Additionally, P₀ ESL colonies were enumerated 3-5 weeks post plating on P₀ MEF cultures with live surface TRA-1-81 antigen immunostaining (StainAlive™ DyLite™488 Mouse anti-Human Tra-1-81 antibody, Stemgent). Reprogrammed cultures were fed with MEF conditioned medium (CM) supplemented with 40 ng mL⁻¹ bFGF after 12 days, and this was continued until AP assays or live TRA-1-81 stainings were performed 3-5 weeks following original nucleofections. Individual ESL subclones were also manually picked from P₀ (CB-iPSC) or P₁ (Fib-iPSC; Ker-iPSC) cultures for expansion and further characterizations.

The completion of reprogramming in bulk populations of emerging hiPSC was determined by FACS analysis of P₀ CM cultures with surface SSEA4, TRA-1-81, and intra-cellular NANOG immunostaining 3-5 weeks following initial MEF platings. Bulk cultures were stained with surface antibodies (BD Biosciences, San Jose, Calif.) for pluripotency markers (SSEA4-APC, TRA-1-60-PE, TRA-1-81-PE) or hematopoietic markers (CD34-PE, CD45-APC, CD34-APC, CD33-PE, CD13-PE). Cells were fixed and permeablized with Fix and Perm kit (Invitrogen) for intracellular NANOG-PE FACS analysis.

Cell Culture.

All tissue culture reagents were purchased from Invitrogen (Carlsbad, Calif.) unless otherwise stated. MEF, hESC and hiPSC culture were maintained at 37° C., 5% CO₂ and 85% relative humidity. Medium was changed daily on hESC and established hiPSC cultures. Pluripotent stem cells were maintained on irradiated mouse embryonic fibroblasts (MEFs) in DMEM/F12 (Invitrogen) medium supplemented with 20% Knockout Serum Replacer (KOSR; Invitrogen), 0.1 mM MEM non-essential amino acids (GIBCO), 0.1 mM β-mercaptoethanol (Sigma) and 4 ng ml⁻¹ FGF2 (R&D systems, Minneapolis, Minn.).

Purified (>95%) human CD34⁺ progenitors from neonatal cord blood (CB), adult bone marrow (BM), and 20-22 week-old fetal liver (FL) were obtained from pooled or individual donors, and purchased from ALLCELLS (Emeryville, Calif.) or Lonza, (Walkersville, Md.). Human mesenchymal bone marrow stromal cells (BMSCs) (Lonza) were cultured in complete MSC medium (Lonza). Keratinocytes were derived from a plucked hair of a normal adult donor, with modified methods as previously described and cultured in a T175 flask coated with EpiLife Coating Matrix and EpiLife Medium with Supplement S7. Fetal fibroblasts harboring the sickle cell mutation (Cat# GM02340), and 56-year old normal female adult skin fibroblasts (Cat# AG07714) were obtained from the Coriell Institute Cell Repository (Camden, N.J.), and cultured in DMEM supplemented with 10% heat-inactivated fetal bovine serum (FBS, HyClone, Thermo Scientific, Waltham, Mass.), 1×MEM non-essential amino acids, 0.1 mM beta-mercaptoethanol, 1 mM L-glutamine and 0.5% penicillin/streptomycin. Keratinocytes and fibroblasts were used at low passages, and freshly passaged 2 to 3 days before nucleofections.

Cell Cycle Analysis.

Cell cycle status of fibroblasts or CD34⁺ CB cells in the presence of hematopoietic GFs (FTK: Flt3L, TPO, kit ligand (SCF)) hematopoietic GFs was determined by EdU incorporation following co-culture for 72 hours with and without BMSC stromal layers. Prior to +/−BMSC culture, CB samples were either mock-nucleofected, or nucloefected with 4F or 7F plasmids on day 0, as described below. CD34+ cells were incubated with EdU 10 uM for 4 hours in FTK medium on Day 0, or 72 hours following nucleofection. Cells were stained with the Click-IT EdU AlexaFluor488 flow kit (Invitrogen, Carlsbad, Calif.) according to manufacturer's instructions, and analyzed on a BD FACScalibur flow cytometer (BD Biosciences, San Jose, Calif.).

Generation of Episomal hiPSC

Plasmids.

The episomal EBNA-based pCEP4 (Invitrogen, Carlsbad, Calif.) vectors pEP4 EO2S EN2L (OCT4, SOX2, NANOG, LIN28), pEP4 EO2S ET2K (OCT4, SOX2, SV40LT, KLF4), pEP4 EO2S EM2K (OCT4, SOX2, MYC, KLF4), pEP4 EO2S EN2K (OCT4, SOX2, NANOG, KLF4), and pEP4-M2L (MYC, LIN28) were obtained from Addgene (Cambridge, Mass.). Plasmids were propagated in TOP10 E. coli (Invitrogen) and purified with QIAGEN plasmid Maxi kits. Ratios of (1:1:1) of each plasmid pCEP4-EO2S-EN2L, pCEP4-EO2S-ET2K, and pCEP4-EO2S-EM2K were mixed as the seven-factor (7F) SOKMNLT “Combo 6”¹. Plasmid pEP4 EO2S EM2K was used singularly for four-factor (4F) SOKM factor nucleofections.

Generation of Non-Integrated Fibroblast- and Keratinocyte-hiPSC.

Fetal fibroblasts (FFB) cells were passaged two to three days prior to nucleofection. Cells were trypsinized, counted, and 1×10⁶ cells were resuspended in 100 μL of nucleofector solution (VCA-1001, Lonza), and a total of 8 μg of the three 7F episomal plasmids, or 4F single episome. The mixture of DNA/cells solution was nucleofected with program U-020 with an AMAXA II nucleofector device. Adult fibroblasts were obtained from a normal 56 year-old donor, and nucleofected in NHDF nucleofector solution (VPD-1001) with 6 μg 7F plasmid mixture per 1×10⁶ cells using program U023. After nucleofection of either fetal or adult fibroblasts, 500 μL of pre-warmed fibroblast medium was added into the cuvette, and the cells were removed immediately and transferred into three 10 cm plates precultured with irradiated MEF. After 4-6 hours incubation the cells were collected, and fresh fibroblast medium was replaced onto the same MEF cultures (P₀). After 72 hours (day 3), the fibroblast medium was replaced with hESC medium containing 40 ng mL⁻¹ FGF2. Adult keratinocytes were similarly prepared and 1×10⁶ cells were nucleofected using Human Keratinocyte Nucleofector Kit (VPD-1002, Lonza, Walkersville, Md.). Keratinocytes were resuspended in 100 μL of Keratinocyte nucleofector solution with of 6 μg of episomal plasmid DNA mixtures and nucleofected with program T-024. After treatment, 500 μL of pre-warmed medium was added into the cuvette, and cells were removed immediately and plated into pre-warmed EpiLife medium with 10% FBS onto MEF feeders. After 4-6 hours incubation, the medium was changed with fresh EpiLife medium. After 72 hours (Day 3), the medium was replaced with hESC medium with 40 ng mL⁻¹ FGF2. For both fibroblast and keratinocytes cultures, cells were fed with MEF-condition medium (CM) containing 40 ng mL⁻¹ FGF2 after Day 10, and passaged onto fresh MEF layers after 3 weeks (P₁).

Generation of Non-Integrated CB-iPSC.

The method of generation of BMSC-primed CB-iPSC, and the derivation and characterization of non-integrated episomal CB-iPS clones 6.2, 6.11, 6.13, and 19.11 were recently described. A schematic for quantitatively evaluating comparative reprogramming efficiencies is summarized in FIG. 9. On Day −3 of the reprogramming protocol, 0.5×10⁶−1.0×10⁶ purified human CD34⁺ progenitors from fetal liver, neonatal cord blood, adult mobilized peripheral blood, and adult bone marrow were thawed, expanded in two ml Stem Span-SFEM medium (StemCell Technologies, Vancouver, BC), and supplemented with FLT3L (100 ng mL⁻¹), and TPO (10 ng mL⁻¹), SCF/Kit ligand (100 ng mL⁻¹), (FTK) (R&D Systems, Minneapolis, Minn.). All reprogramming culture steps were conducted in tissue culture plates that were tightly wrapped in Saran wrap for induction of hypoxic conditions. After three days (on day 0), cells were collected by centrifugation (200 g, 10 min) and counted. 0.5×10⁶⁻1.0×10⁶ CD34 progenitors were nucleofected with 6 μg total of 4F or combined 7F plasmid DNA (combination 6 or 19, as above) using the AMAXA II nucleofector device (Lonza), program U-008, and 100 μL CD34⁺ nucleofector solution VPA-1003 (Lonza). Following nucleofection, 500 μL of pre-warmed medium was added into the cuvette, and cells were replated immediately into one mL pre-warmed RPMI 1640 medium with 10% FBS in a 12 well plate. After 4-6 hours incubation in RPMI/10% FBS, nucleofected CD34⁺ cells were collected and replated onto Retronectin (Takara Bio, Madison, Wis.)-coated (10 μg mL⁻¹) E-well plates seeded with confluent, irradiated (2000 cGy) human mesenchymal bone marrow stromal cell (BMSC) feeders. Nucleofected Day 0 CB progenitors were expanded in these BMSC co-cultures in SFEM supplemented with 100 ng mL¹ FLT3L, 50 ng TPO, and 100·ng SCF (FTK GFs). Three days later (Day 3), CB or CB-BMSC cultures were harvested enzymatically, and single viable CB cells were counted, and 300-20,000 cells were replated onto irradiated MEF feeder plates in 2 mL SFEM containing FTK GFs, as above. On Day 4, two mL of hESC medium containing 40 ng FGF2 was added to MEF cultures. On Day 6, and every 2 days thereafter, one-half the medium volume in each well was harvested (hemidepletion), and hematopoietic suspension cells were returned into their respective wells with 2 mL fresh hESC medium containing 40 ng mL⁻¹ FGF2 (i.e., gradually tapering the concentration of FTK GFs from Day +3). Starting on Day 12, MEF-conditioned medium (CM) supplemented with 40 ng FGF2 was used for subsequent medium changes. ESC-like (ESL) colonies emerged with these conditions with CD34⁺ progenitors as early as 7-10 days post-nucleofection.

Determination of Reprogramming Efficiencies of FACS-Purified Hematopoietic Populations.

Episomal Reprogramming of FACS-Purified CD34⁺CD38^{hi} and CD34+CD38^{low} Hematopoietic Populations.

A schematic that summarizes the reprogramming strategy of FACS-purified populations is included in FIG. 2. Highly purified (>96%) CD34⁺CD45⁺ CB cells were obtained commercially (AllCells), and thawed according to manufacturer's instructions. CD34⁺ CB cells were cultured initially (day −3; FIG. 9) in Stem Span-SFEM medium (StemCell Technologies, Vancouver, BC) supplemented with FLT3L (100 ng mL⁻¹), and TPO (10 ng mL⁻¹), and SCF/Kit ligand (100 ng mL⁻¹), (FTK) (R&D Systems, Minneapolis, Minn.) overnight. The next day (day −2), viable cells were collected for FACS purification in Stem Span-SFEM medium and centrifuged in 200 g, 5 min. CD34⁺ cells were stained with mAb CD38-APC (BD Biosciences) for 30 min on ice. FACS gates for both CD38^{high} and CD38^{low}-expressing cells (19.4±7.39%, and 19.73±7.24% respectively, n=3) were identified, and purified CD34⁺ populations were collected in SFEM medium containing FTK GF (as above), and cultured an additional two days (until day 0; FIG. 9). On day 0, FACS-purified CD34⁺CD38^{hi} and CD34⁺CD38^{1ow} populations were nucleofected with a single episome expressing 4F (pEP4 EO2S EM2K; see below), and cultured further in GF +/−BMSC co-culture, exactly as described above, for an additional 3 days (FIG. 9). Single CB cells from each purified population were plated on MEF on day 3 for subsequent reprogramming efficiency determinations, exactly as described above for unsorted CB cells.

Reprogramming of FACS-Purified Lineage-Committed Myeloid Progenitors Expressing Reprogramming Via GFP Co-Expression.

A schematic that summarizes the reprogramming strategy of FACS-purified populations enriched for expression of reprogramming episomes is included in FIG. 2. For these experiments, highly purified (>96%) CD34⁺CD45⁺ CB cells were received within 24 hours of neonatal harvest from AllCells (catalog number: CB005). On the same day (day −3 of the reprogramming protocol; FIG. 9), CB cells were plated in 2 mL of Stem Span-SFEM medium (StemCell Technologies, Vancouver, BC) supplemented with hematopoietic GFs: FLT3L (100 ng mL⁻¹), TPO (10 ng mL⁻¹), and SCF/Kit ligand (100 ng mL⁻¹; all R&D Systems, Minneapolis, Minn.). Culture plates were tightly wrapped in Saran wrap to create hypoxic cultures. On day 0, GF-activated myeloid progenitors were collected in Stem Span-SFEM medium, and centrifuged at 200 g for 5 min. 0.5×10⁶ CD34 progenitors were nucleofected with 6 μg of 4F-plasmid DNA (pEP4 EO2S EM2K) and 2-3 μg of pEP4-EF1a-eGFP (with same vector backbone and promoter as the 4F episomal construct) using the AMAXA II nucleofector device (Lonza). Program U-008, and 100 μL CD34⁺ nucleofector solution VPA-1003 (Lonza) was employed. Following nucleofection, 500 μL of pre-warmed medium was added into the cuvette, and cells were replated immediately into one mL pre-warmed RPMI 1640 medium with 10% FBS in a 12 well plate. After 4-6 hours, nucleofected CB cells were collected, centrifuged, resuspended in Stem Span-SFEM medium with FLT3L, TPO, SCF/Kit ligand (100, 50,100 ng mL⁻¹, respectively), and plated onto Retronectin (Takara Bio, Madison, Wis.)-coated (10 μg mL⁻¹) 12-well plates, or onto irradiated (2000 cGy) BMSC feeder layers that were similarly pre-coated with Retronectin. Three days later (Day 3), CB cells were harvested, and stained with CD34-PE (BD BioSciences) antibody for 20 min on ice. BMSC were easily distinguished from CB cells by forward scatter and side scatter gates, and excluded for cell sorting. Three populations of Day 3 CB cells were purified based on GFP expression: GFP⁻CD45⁺ (non-transgene-expressing cells), GFP⁺CD34⁺CD45+, and GFP⁺CD34⁻CD45+ expression (transgene-expressing cells). These sorted GFP⁺ and GFP⁻ CB populations were plated onto MEF on day 3, and reprogramming efficiencies determined, as above.

Flow Cytometry, Immunocytostaining, AlkalineP, and Live Tra-1-81 Surface Antigen Staining.

Flow Cytometry.

hESC and hiPSC cultures were dissociated enzymatically, passed through a 40 μm filter to remove cellular debris, and then centrifuged for 5 min at 200 g. The cells were gently resuspended in PBS containing 5% FBS, and stained with monoclonal antibodies for 30 min on ice. Antibodies included APC conjugated SSEA4 (R&D Systems), PE Mouse anti-Human Tra-1-60 antigen (BD Biosciences) and PE Mouse anti-Human Tra-1-81 antigen (BD Biosciences). For intracellular OCT3/4, SOX2, and NANOG FACS staining, cells were fixed and permeabilized using FIX & PERM Cell Permeabilization Reagent (Invitrogen), and the cells were stained with anti-human/mouse OCT3/4-PE (R&D Systems), SOX2-PE, or mouse anti human Nanog-PE (BD Biosciences). Cells were washed in 5% FBS/PBS and analyzed using a FACSCalibur instrument (BD Biosciences). Data were analyzed using FLOWJO flow cytometry analysis software (www.flowjo.com).

hiPSC Colony Enumeration by Alkaline Phosphatase (AP) Staining and Live TRA-1-81 Staining.

hiPSC cultures were fixed in 4% paraformaldehyde/PBS for 10 minutes, and washed in 1×PBS and stained with AP substrate in 1 step NPT/BCIP reagent (Sigma) for 10 to 15 min at room temperature. The reactions were stopped after 15 minutes, and wells were washed three times with 1×PBS. Only colonies that stained strongly and within 15 minutes (AP^{hi}) were enumerated. In alternate replicate wells, TRA-1-81 StainAlive Dylight 488-conjugated antibody (1:100; Stemgent, Cambridge, Mass.) was diluted in hESC medium and directly added into P₀, and later in P₁ iPSC cultures. After 30 min, cultures were washed twice with hESC medium, and TRA-1-81 positive colonies were visualized with fluorescence microscopy. Three to five weeks following episomal nucleofections, ESL colonies were counted and stained live with TRA-1-81 fluorescent antibodies on the original of Pₒ MEF cultures, and fluorescent colonies were enumerated.

mRNA Expression, Bioinformatics Data, and Gene Set Enrichment Analysis (GSEA) Analysis.

Collection of Cell Samples for Expression Microarrays.

Bulk reprogrammed cultures were collected from BMSC (day 3) or MEF co-cultures (day 23) and filtered through a 40 μm cell-strainer. Samples were the further purified by FACS sorting on viability (day 23 samples) or CD45⁺ expression (for day 3 samples). FACS-purified cells were kept on ice until centrifuged and snap frozen in liquid nitrogen for RNA purification and subsequent Illumina gene array analysis. All hESC/iPSC lines were confirmed to be >98% SSEA4⁺Tra-1-60⁺Tra-1-81⁺ by FACS prior to harvesting cell pellets for RNA to be used in qRT-PCR or Illumina gene microarrays. All pluripotent stem cell lines were passaged from MEF onto Matrigel and expanded with MEF-conditioned medium (CM) for one passage prior to harvesting cells for expression studies to remove irradiated MEF.

Gene Expression Microarrays.

Human HT-12 Expression BeadChip arrays (Illumina, San Diego, Calif.) were used for microarray hybridizations to examine the global gene expression of hESC, hiPSC, and starting populations (CD34⁺ progenitors, keratinocytes, and fibroblasts). Each array on the HumanHT-12 Expression BeadChip array targeted more than 25,000 annotated genes with more than 48,000 probes derived from the National Center for Biotechnology. Information Reference Sequence (NCBI) RefSeq (Build 36.2, Rel 22) and the UniGene (Build 199) databases. Total RNA was prepared as described in the RNeasy Mini Kit (QIAGEN) with on-column DNase I digestion. All samples were processed at the Sidney Kimmel Comprehensive Cancer Center Microarray Core Facility at Johns Hopkins University, Baltimore. Briefly, 200 ng total RNA from each sample was amplified and labeled using the Illumina TotalPrep RNA Amplification Kit, AMIL1791 (Ambion, Austin, Tex.) as described in the manufacturer's instruction manual. All arrays were hybridized at 58° C. for 16-20 hours followed by wash and stain procedures according to the Whole-Genome Gene Expression Direct Hybridization Assay Guide (Illumina, San Diego, Calif.). Fluorescent signals were obtained by scanning with the iScan System and data were extracted with Gene Expression Module 1.0.6 in GenomeStudio 1.0.2 and signal intensities from multiple chips were normalized without background subtraction.

Expression Arrays Bioinformatics Data Analysis.

Gene expression data from the Human HT-12 arrays, described above, were analyzed with the Partek Genomics Suite (Partek Inc., St. Louis, Mo.) and Spotfire DecisionSite for Functional Genomics™ (TIBCO Software Inc., Somerville, MA) platforms. The scanned fluorescent signal data were quantile normalized in Illumina Bead Studio to allow cross array comparison, and were then imported into Partek where they were first log 2 transformed for analysis. Log 2 signal values were normalized by subtracting each gene's mean value prior to clustering in order to represent expression change across cell type, rather than overall signal intensity, and indicate the cell lines' similarity and correlation. For heat maps presented, these expression values were mean-normalized to better demonstrate how gene expression differed across the examined cell types. In mean normalization, each gene's mean log 2 signal value is determined for all cell types and then subtracted (division in log space) from each cell type's value for that gene. The normalized values underwent unsupervised hierarchical clustering in Spotfire (Euclidean distance algorithms) to compare cell types' gene expression in a heat map-dendrogram wherever indicated (color spectrum indicates where lower=blue (or solid); higher=red (or striped). The R² values shown are the square of the Pearson R correlation coefficient between the two cell types' correlation, where higher value indicates greater correlation (all R values were positive). Partek software was used to compare the mean normalized log 2 signal values of pluripotency-associated gene modules (e.g. ESC core, MYC, PRC1, PRC2, Core) in box and whisker plots, and Spotfire to determine the Pearson R correlation coefficient (PCC) between cell types' log 2 expression values. Finally, Spotfire software was used to construct scatterplots. These scatterplots compare genes relative expression levels between two classes of cell lines, depicting each gene's log 2 fold-change between classes on the Y-axis and its average log 2 value (or methylation beta values) for cell types on the X-axis.

Gene Set Enrichment Analysis (GSEA).

Significantly expressed gene sets were determined from normalized Illumina array data using the GSEA computational method (http://www.broadinstitute.org/gsea). The GSEA method determines whether an a priori defined set of genes shows statistically significant, concordant differences between two biological states. The set of genes that were statistically significantly changed (t test, p<0.05) between two experimental conditions of interest were identified using multivariate ANOVA. GSEA was performed on these sets of genes using GSEAP v2.07 (http://www.broad.mit.edu/gsea) using the MSigDB v. 3.0 Reactome gene sets, with an FDR<0.05 as threshold for significance.

Proteomic Studies of CB-BMSC Co-Culture Supernatants.

Media supernatants were harvested from Day 3 CB cells that had been co-cultured with or without irradiated BMSC layers for 3 days in SFEM-FTK (Flt3L, TPO, SCF) and Retronectin in conditions exactly as for reprogramming experiments. Supernatants were frozen at −80 C. Supernatants were later analyzed by antibody arrays (L-series glass chip antibody array, RayBiotech, Norcross, Ga.). Raw intensity values from array analysis were normalized to positive controls and background subtracted. Expression of molecules was normalized and ranked based on the ratio of their expression in BMSC-conditioned vs. non-conditioned media.

Teratoma Assays.

Low passage hiPSC lines were passaged from MEF onto Matrigel cultures and expanded with MEF-conditioned medium (CM) prior to harvest and teratoma injections. Briefly, hiPSC were grown to 60-80% confluency on Matrigel/CM, harvested as clumps with collagenase TV (Invitrogen), resuspended in a mixture of hESC medium and Matrigel (BD Biosciences) at a ratio of 1:1, and ˜10⁷ cells were injected intramuscularly (hind leg) into immunodeficient NOG SCID mice (approximately two 6-well plates per mouse). After six to twelve weeks, teratomas were dissected, fixed in 4% paraformaldehyde, embedded in paraffin, and stained with hematoxylin and eosin.

Karyotypes of Pluripotent Stem Cell Lines.

Karyotyping was performed by high resolution O-banding at the JHUSM Cytogenetics Core.

Polymerase Chain Reaction (PCR).

Reverse Transcriptase (RT) and Genomic PCR.

RT-PCR analysis for transgene expression and EDNA1 vector backbone were performed with primers as described. See Burridge et al., 6(4) PLoS ONE e18293. doi:10.1371/journal.pone.0018293 (2011); and Yu et al., 324 SCIENCE 797-801 (2009). Briefly, total RNA was extracted from passage 11 CB-iPSC clones, negative control passage 48 H9 hESC, and positive control “bulk” (passage 2) early CB-iPSC that were nucleofected with episomal vectors (˜14-21 days old) using the RNeasy Mini Kit (QIAGEN). cDNA was generated from each sample using SuperScript-First Strand Synthesis (Invitrogen), and PCR reactions were performed with Pfx DNA polymerase (Invitrogen) using the protocol described previously. PCR products were analyzed on 2% agarose quick gels (Invitrogen). Genomic and episomal DNA were extracted from passage 11 CB-iPSC, negative control H9 hESC, and positive control bulk CB pre-iPSC using DNeasy Blood & Tissue Kit (QIAGEN). Genomic PCR reactions were performed with Pfx DNA polymerase as described in Yu et al., 2009. PCR products were analyzed on 2% agarose gels.

Quantitative Real-Time RT-PCR (qRT-PCR).

Total RNA from all hiPSC/hESC or donor cell samples was prepared using the RNeasy Mini Kit with on-column DNase I digestion (QIAGEN). First-strand cDNA was reverse transcribed with oligo-dT using SuperScript First-Strand (Invitrogen). qRT-PCR was performed using iQ SYBR-Green (BioRad, Hercules, Calif.) or Power SYBR PCR Mastermix (Applied Biosystems, Foster City, Calif.) and ABI thermal cycler and software. Human-gene specific PCR amplicons of 90-300 bp (see PCR Primers table below) were designed with PRIMER 3.0 software (http://frodo.wi.mit.edu/primer3/), and all primers were optimized for the following conditions: initial denaturation for 5 min at 95′C; 45 cycles of 95° C. 15 sec, 60° C. 30 sec, 68° C. 30 sec. Transcripts of target genes and beta actin controls for each cDNA sample were amplified in triplicates/quadruplicates. All qRT-PCR reactions were confirmed for specificity of a single PCR product by analysis on 4% agarose quick gels. Relative qRT-PCR analysis using the 2^{-ΔΔT} method was performed using cycle threshold (C_{T}) normalized to beta actin as described. Fold change expression of actin-normalized CB-iPSC clones was compared to control H9 hESC. For the analysis of endogenous gene expression of nucleofection target cells for iPSC formation, HSC GF-activated CB (AllCells or Lonza) were thawed, expanded for 3 days in Flt3L(100 ng/ml), TPO (10 ng/ml) and SCF (100 ng/ml), and used for RNA and cDNA preparation followed by qRT-PCR analysis relative to control H9 hESC, as described above.

The following PCR Primers were used in these studies to evaluate transgenic (episomal) and endogenously-expressed pluripotency genes. See Peters et al., Human Embryonic and Induced Pluripotent Stem Cells, Springer Protocols Handbooks, Part2: 202-227. DOI: 10.1007/978-1-61779-267-0_{—}16 (2011).

TABLE 1
PCR Primer

| Human- | | | Amplicon | |
|---|---|---|---|---|
| Specific | Forward Primer | Reverse Primer | Size, | Annealing |
| Genes | 5′ to 3′ | 5′ to 3′ | base pairs | Temp. ° C. |
| Transgene | | | | |
| Genomic- | | | | |
| PCR, see | | | | |
| Yu et al, | | | | |
| 2009 | | | | |
| Transgene | | | | |
| RT-PCR, see | | | | |
| Yu et al., | | | | |
| 2009 | | | | |
| Endogenous | | | | |
| Genes qRT- | | | | |
| PCR | | | | |
| ACTIN | GGC ATC CTC ACC | GGG GTG TTG AAG | 203 | 60 |
| | CTG AAG TA | GTC TCA AA | | |
| | (SEQ ID NO: 1) | (SEQ ID NO: 2) | | |
| SOX2 | CCC AGC AGA CTT | CCT CCC ATT TCC | 151 | 60 |
| | CAC ATG T | CTC GTT TT | | |
| | (SEQ ID NO: 3) | (SEQ ID NO: 4) | | |
| OCT3/4 | CCT CAC TTC ACT GCA | CAG GTT TTC TTT | 164 | 60 |
| | CTC TA | CCC TAG CT | | |
| | (SEQ ID NO: 5) | (SEQ ID NO: 6) | | |
| KLF4 | GAC CAC CTC GCC | TGG GAA CTT GAC | 161 | 60 |
| | TTA CAC AT | CAT GAT TG | | |
| | (SEQ ID NO: 7) | (SEQ ID NO: 8) | | |
| C-MYC | AAG AGG ACT TGT | CTC AGC CAA GGT | 179 | 60 |
| | TGC GGA AA | TGT GAG GT | | |
| | (SEQ ID NO: 9) | (SEQ ID NO: 10) | | |
| NANOG | CTC CAT GAA CAT | GGCATC ATG GAA | 157 | 60 |
| | GCA ACC TG | ACC AGA AC | | |
| | (SEQ ID NO: 11) | (SEQ ID NO: 12) | | |
| LIN28 | CAC AGG GAA AGC | TGC ACC CTA TTC | 162 | 60 |
| | CAA CCT AC | CCA CTT TC | | |
| | (SEQ ID NO: 13) | (SEQ ID NO: 14) | | |
| UTF1 | AGC TGC TGA CCT | GTG GGA AGG CAG | 204 | 60 |
| | TGA ACC AG | CAG GAG | | |
| | (SEQ ID NO: 15) | (SEQ ID NO: 16) | | |
| ABCG2 | AGC TGC AAG GAA | TCC AGA CAC ACC | 286 | 60 |
| | AGA TCC AA | ACG GAT AA | | |
| | (SEQ ID NO: 17) | (SEQ ID NO: 18) | | |
| REX1 | TTT ACG TTT GGG | TCT GTT CAC ACA | 92 | 60 |
| | AGG AGG TG | GGC TCC AG | | |
| | (SEQ ID NO: 19) | (SEQ ID NO: 20) | | |
| DNMT3B | CTA CTG CCC AGC | TTG CAC CCA GGA | 149 | 60 |
| | ATG TCA GA | TCC TTA AC | | |
| | (SEQ ID NO: 21) | (SEQ ID NO: 22) | | |
| TP53 | GGC CCA CTT CAC | GTG GTT TCA AGG | 156 | 60 |
| | CGT ACT AA | CCA GAT GT | | |
| | (SEQ ID NO: 23) | (SEQ ID NO: 24) | | |
| hTERT | GCC GTT TCC AAA | AAC ACC TAG CAT | 294 | 60 |
| | CAC AGA GT | GGG TGA GG | | |
| | (SEQ ID NO: 25) | (SEQ ID NO: 26) | | |

### Results

### Example 1

### Brief Expansion of GF-Activated CD34⁺ CB Cells on Irradiated BMSC Prior to Episomal Reprogramming Enhanced Hematopoietic Frequency and Viability Without Affecting Proliferative Capacity

The present inventors report herein the derivation of non-integrated, transgene-free CB-derived hiPSC lines (CB-iPSC) that were generated at high efficiencies (−1-4% of input cells) using a novel BMSC co-culture system and a seven-factor EBNA-based episomal system (7F; SOX2, OCT4, KLF4, MYC, NANOG, LIN28, and SV40 T antigen; SOKMNLT'). See Burridge et al., 6(4) PLOS ONE e18293. doi:10.1371/journal.pone.0018293 (2011). In designing this reprogramming system (FIG. 9a), the present inventors capitalized on the principle that the innate epigenetic plasticity of hematopoietic progenitors can be positively influenced by stem cell niche signals. This optimized BMSC co-culture system provided soluble factors that preserved the short-term viability, as well as frequencies of CD34⁺CD45⁺ multipotent erythro-myeloid hematopoietic progenitors (e.g., GEMM-CFU and BFU-e) following plasmid nucleofection, compared to continued culture with hematopoietic GFs alone (FIGS. 1a, 10a-b). However, BMSC co-culture did not increase the percentage of CB progenitor cell proliferation compared to GF stimulation alone, since the percentages of CB cells entering into S phase compared to short-term expansion with hematopoietic GFs alone were similar, and also comparable to cultured adult fibroblasts (FIG. 1b).

### Example 2

### Episomal Reprogramming was Strikingly Rapid and Efficient in BMSC-Primed CB Progenitors, and Unlike Fibroblasts or Keratinocytes Required Only the Four Yamanaka Factors

To define conditions for optimized hematopoietic progenitor reprogramming, the hypothesis that a stromal micro-environment that enhances hematopoietic self-renewal would also augment the episomal reprogramming efficiency of CB progenitors was first tested. Highly purified (>96% CD34⁺CD45⁺) CB progenitors were activated with hematopoietic growth factors (GF; FIG. 9a) followed by nucleofection with plasmid episomes expressing defined factors. Day 0 GF-activated CB populations contained few primitive CD34⁺CD38⁻ stem-progenitors, and consisted predominantly (i.e., >95% CD34⁺CD38⁺, and >99% CD33⁺CD45⁺) lineage-committed progenitors on the day of nucleofection (FIGS. 10a, 10c). Day 0 CB progenitors were nucleofected with a single pulse of either four (4F; SOX2, OCT4, KLF4, MYC: ‘SOKM’) or seven (7F; SOKMNLT) episomal factors and subsequently co-cultured with or without irradiated human mesenchymal BMSC for an additional 3 days (FIG. 9a). This was followed by quantitative plating of single CB cells on MEF (on day 3) for reprogramming efficiency determinations. The episomal 4F and 7F reprogramming efficiencies of +/−BMSC-primed CD34⁺ CB progenitors were comparatively evaluated in parallel experiments against adult keratinocytes, fetal fibroblasts, or adult fibroblasts 3-5 weeks later (FIG. 9b). Pilot gene transfer experiments with GFP constructs revealed that poor nucleofection gene transfer efficiency of CB and fibroblasts of the extremely large (−15-18 kb) episomal constructs was a limiting factor (in the range of ˜10-20%; FIG. 11) in the reprogramming protocol, and may partially account for the poor efficiencies of episomal reprogramming previously reported.

Rare, tightly-packed ESC-like colonies with sharply defined borders emerged from 7F-nucleofected keratinocytes and fetal/adult fibroblasts with extremely low efficiencies (<0.001%) and slow kinetics (˜5-7 weeks following gene transfer). The majority of episomal fibroblast-iPSC clones that emerged either did not expand, or unstably differentiated following 1-2 subcloning passages. In striking contrast, 7F and 4F-nucleofected CB generated hiPSC colonies with marked rapidity (7-21 days following a single episomal nucleofection pulse), and the majority (>90% of clones) maintained a stable, proliferative ESC-like morphology that permitted subsequent manual subcloning with minimal effort. In greater than 10 independent experiments using pooled donor CB, BMSC priming reproducibly augmented the generation of 7F and 4F episomal CB-iPSC colonies with significantly higher efficiencies (p<0.05) that were ˜10,000-fold greater than any previously reported episomal reprogramming method for fibroblasts (FIG. 1c). Furthermore, unlike fibroblasts, which did not yield episomal hiPSC with less than 7F, 4F SOKM reprogramming of CD34⁺CD45⁺CB progenitors with a single pulse of one episomal plasmid (pCEP4-EM2K) was even more robust than with equimolar DNA quantities of the 7F three-plasmid system. Reprogrammed Type III CB-iPSC emerged rapidly with 4F from BMSC-primed CD34⁺CB cells (as early as 7-14 days) at significantly higher frequencies (20-50× fold higher; p<0.05) than without BMSC co-culture (5-20% reprogramming efficiencies per input cell). Moreover, BMSC-primed 4F CB cells generated bulk populations of CB-iPSC cultures that were phenotypically fully-reprogrammed (i.e., 50-80% NANOG⁺Tra-1-81⁺and >80-95% SSEA4⁺) (FIG. 1c). Because the episomal nucleofection efficiency of Day 0 CB cells was in the range of 10-20% (FIG. 11), these unprecedented reprogramming efficiencies of up to ˜20% suggested the possibility that BMSC-priming may actually accelerate the reprogramming of the majority of successfully SOKM-nucleofected BMSC-primed CB progenitors with efficiencies much higher than previously reported.

### Example 3

### Lineage-Committed Myeloid Progenitors, and not Hematopoietic Stem-Progenitors were More Efficient Targets of Episomal Reprogramming

Previous studies suggested that stem-progenitors have an augmented propensity for pluripotency induction relative to more differentiated somatic targets. To determine the true reprogramming potential of hematopoietic cells in our system, whether rare stem-progenitors within heterogeneous CB populations were more amenable to reprogramming than lineage-committed progenitors was tested. Thus, CD34⁺progenitors were FACS-purified at the initiation of the reprogramming protocol (day −2) into stem-progenitor-enriched (CD34⁺CD38⁻) or lineage-enriched (CD34⁺CD38⁺) fractions (i.e., prior to Day 0 4F nucleofections and +/−BMSC priming (FIG. 2a). Post-sort FACS analysis verified that this purified Day −2 CD34⁺CD38⁺fraction consisted of >95%-enriched CD33⁺CD13⁺myeloid progenitors. These experiments surprisingly revealed that the lineage-enriched (CD34⁺CD38⁺) fraction reprogrammed significantly more rapidly (16.73%+/−3.7 input efficiency; range: 13.0-20.4%, n=2) than the more primitive CD34⁺CD38⁻stem-progenitor fraction (0.33%+/−0.30 input efficiency). MEF wells that were seeded with as few as 2000 BMSC-activated CD34⁺CD38⁺-selected CB cells nucleofected with 4F routinely generated cultures containing 120-300 AP⁺CB-iPSC colonies. Furthermore, these P₀ cultures routinely produced >5×10⁶ fully reprogrammed cells that had acquired expression of NANOG and pluripotency surface markers TRA-1-81 and TRA-1-60 in >60-80% of all cells by 3-4 weeks following 4F nucleofection.

### Example 4

### CD34-Negative Myeloid Cells Expressing Episomal Factors Reprogrammed with ˜50% Efficiency

A study that utilized transgenic mice expressing the Yamanaka factors homogenously in all somatic donor cells reported that hematopoietic stem and progenitor cells could be reprogrammed with efficiencies as high as 8-28%. Because nucleofection gene transfer was limiting in the present episomal system (˜10-20%; FIG. 11), it was hypothesized that reprogramming in successfully-nucleofected cells may actually be higher than the ˜20% efficiencies observed for lineage-committed CB cells. To determine the true reprogramming efficiency of myeloid progenitors, a strategy (see schematic FIG. 2) was employed that simultaneously enriched for both lineage-committed CB cells, and CB cells that had been successfully nucleofected with a single pulse of the large 4F episomal plasmid. CB cells were first co-nucleofected on day 0 with a parental pCEP4-GFP construct along with the 4F episome (pEP4 EO2S EM2K). After three days of +/−BMSC co-culture, CD34-positive and CD34-negative episome-expressing progenitors were isolated by FACS-purification. Three CB populations were sorted based on GFP and CD34 expression: GFP⁻, GFP⁺CD34⁺, and GFP⁺CD34⁻expression (FIG. 2b). These sorted CB populations were then plated onto MEF on Day 3 for AP⁺ESL colony reprogramming efficiency determinations, as above. Unexpectedly, these experiments revealed that CD34-negative (CD33⁺CD45⁺) myeloid cells, and not CD34-positive progenitors generated dramatically higher frequencies of AP⁺TRA-1-81⁺ESL colonies. Under these conditions, at least 50% of episome-expressing (GFP+) CD34⁻CD45⁺BMSC-primed lineage-committed CB cells rapidly converted to a pluripotent state. Taken together, these data collectively demonstrated, for the first time, that the four Yamanaka factors expressed on a single non-integrating plasmid were sufficient to efficiently reprogram a large majority of episome-expressing human myeloid populations without need for additional oncogenic factors (e.g., SV40T Ag or LIN28), repeated transfections, or mutagenic chromatin-modifying small molecules.

### Example 5

### GF-Activated Myeloid Progenitors Did not have Increased Endogenous Expression of Reprogramming or Core Factors, but Abundantly Expressed ESC-Like Epigenetic Regulatory Circuits

The present inventors next sought to identify the factors that mediated highly efficient pluripotency induction from myeloid progenitors. High endogenous expression of key core factors (e.g. SOX2) was previously suggested to account for the relative ease of reprogramming observed in neural stem cells. Quantitative real-time RT-PCR analysis of various donor populations revealed that endogenous MYC, and KLF4 were expressed 6-30×-fold higher in Day 0 hematopoietic progenitors (e.g., FL, CB, mPB, BM, and CD34⁺CD38^{+/lo} sorted CB) compared to fibroblasts, but at similar levels compared to keratinocytes (FIG. 3a-b). However, these studies did not reveal decreased p53 expression, or increased expressions of known ESC-specific reprogramming factors (e.g., SOX2, OCT4, LIN28, UTF1, NANOG, etc) in CB cells that could account for the dramatic differences in reprogramming efficiencies observed between fibroblasts, keratinocytes, and CB progenitors.

To gain further insight, the focus was shifted from pluripotency-associated factors to transcriptional circuits known to direct efficient induction of pluripotency. The expression of known pluripotency-associated networks at sequential stages of CB reprogramming were evaluated via microarray analysis and a modular bioinformatics approach. In preliminary analyses, it was found that in contrast to adult fibroblasts, GF-activated CB progenitors expressed a broad palette of chromatin remodeling factors that are known to experimentally enhance iPSC generation (e.g. members of the MYC, Polycomb (PRC2, PRC1), Chromodomain (CHD), SWI/SWF, and Trithorax complex families) (FIG. 12). These transcription factor complexes regulate the upper tier of stem-progenitor networks that regulate epigenetic plasticity, self-renewal, and lineage specification in both hematopoietic and pluripotent stem cells. These factors were expressed in GF-activated Day 0 CB cells at mean levels that were even higher than hESC. The networks these factors regulate include the MYC complex-regulated transcriptional circuits (e.g., the ‘ESC module’, and the recently described MYC module), as well as the lineage-repressive bivalent Polycomb group (PcG) circuits (i.e., PRC1, PRC2 modules) (FIG. 19/TABLE S1).

### Example 6

### ESC-Like MYC and Polycomb (PcG) Circuits were Expressed in De Novo Partially Reprogrammed States, and were Rapidly Reconfigured from Hematopoietic to ESC-Like Transcriptional Patterns Following Episomal Reprogramming

These data suggested an alternative etiology for efficient reprogramming of CB myeloid progenitors: conversion to pluripotency was facilitated not by endogenous somatic expression of key ESC-specific factors, but by a molecular infrastructure of poised pluripotency-associated regulatory circuits (e.g., ESC, MYC, PRC1, PRC2 modules). Thus, the present inventors next sought to correlate the modular expressions of these networks to the observed 4F reprogramming efficiencies of CB progenitors and fibroblasts. Module expressions were quantified before and after 4F expression in donor fibroblasts and CB progenitors at sequential phases of reprogramming (D-3, D0, and +/−BMSC-primed D3 samples), as well as in newly emerged Day +23 bulk CB-iPSC cultures (which consisted of majority populations of NANOG+ cells (FIG. 2). These analyses revealed that relative to fibroblasts and Day −3 naïve GF-unprimed CD34⁺CB cells, Day +3 GF-activated CB progenitors expressed strongly active ESC-like MYC-regulated modules (MYC, ESC), and ESC-like inactive Polycomb complex (PcG)-regulated (PRC1, PRC2) modules (FIG. 4a). Although D0 to D3 CB progenitors possessed a transcriptionally inactive Core module, the mean expression levels of hematopoietic ESC, MYC, PRC1, and PRC2 modules were already comparable to levels in pluripotent stem cells. In contrast, fibroblasts did not possess ESC-like levels of expression for any of these pluripotency circuits.

The composite modular expression patterns of activated D0-D3 CB progenitors was identical to the previously described ‘partially-reprogrammed’ iPSC state that consisted of activated ESC-like expression levels of MYC- and inactivated PcG-regulated modules, but required only activation of the Core module to complete somatic induction to a stable pluripotent state. Collectively, these experiments revealed several important principles regarding CB reprogramming: 1) GF stimulation alone activated MYC-regulated modules (ESC, MYC) to ESC-like levels without significantly affecting Core module expression or PcG module expression (which was already in an ESC-like inactive state in CB cells); 2) these pre-activated pluripotency-associated circuits rapidly reconfigured from hematopoietic to ESC-like patterns (including ESC and Core modules), as observed in early day 23 bulk cultures of CB-iPSC following ectopic 4F expression and stromal co-culture (FIG. 4b). More importantly, this highly efficient reprogramming system ultimately produced high quality non-integrated CB-iPSC lines with normal karyotypes and transcriptional signatures by microarray that were more akin to hESC at low passages (p9-12) than non-integrated fibroblast- and keratinocyte-iPSC (FIGS. 13-17).

### Example 7

### Reprogramming Efficiency of Somatic Donor Cells Correlated Directly to Levels of ESC-L Regulatory Networks and an Activated OCT4-Associated Circuit

The present inventors' observation that GF-activated hematopoietic progenitors already expressed multiple active ESC-like circuits and epigenetic remodeling factors posed the possibility that a wider and more organized pluripotency-associated framework existed in hematopoietic cells that may be directly responsible for facilitating myeloid reprogramming. For example, the critical core pluripotency factor OCT4 is known to physically interact not only with its core factor partners (e.g. SOX2 and NANOG), but also with a known, defined supportive network (the ‘OCT4 interactome’) that regulates transcription, DNA repair, DNA metabolism, and chromatin modification (e.g., PRC1, SWI/SWF, NuRD, CHD, Trithorax complexes). Using a modular approach, as above, the transcriptional activity of this OCT4-associated circuit was measured, as well as several other epigenetic regulator families that experimentally enhance iPSC generation and maintain the pluripotent state (e.g., MYC and PRC2 complex regulators; FIG. 19/TABLE S1). Strikingly, in contrast to fibroblasts and naïve un-stimulated CB cells, GF-activated CB progenitors robustly over-expressed this OCT4-associated network (FIG. 5a) including its epigenetic regulator component (FIG. 5b), as well as the MYC and PRC2 complexes, which have been experimentally validated to be indispensable for pluripotency and somatic reprogramming (FIGS. 5c, 5d). These data further validated the working hypothesis that ESC-like networks, including poised OCT4-interacting circuits, are not ESC-specific but likely regulate similar processes of self-renewal and lineage specification in both hematopoietic progenitors and ESC.

To determine if expression of these networks directly correlated to reprogramming efficiency, the expression levels of these ESC-like modules was next quantitated at progressive developmental stages of donor cells. GF-activated Day 0 progenitors from progressive stages of CD34⁺developmental maturity (i.e., 20-22 week-old fetal liver (FL), neonatal CB, adult GCSF-mobilized peripheral blood (mPB), or adult bone marrow (BM) as well as fibroblasts and keratinocytes were first assayed for their comparative reprogramming efficiencies (FIG. 6a). Regardless of hematopoietic source, ESL colonies with high AP staining and surface Tra-1-81 expression were generated at significantly higher efficiencies compared to episomally-nucleofected keratinocytes and fibroblasts. However, the efficiency of ESL colony generation correlated exactly with the developmental stage of the hematopoietic progenitor, with a hierarchy of reprogramming rate and efficiency: FL>CB>adult mPB>adult BM. Strikingly, a computation of the expression across all of the somatic targets of the MYC complex and its downstream targets (ESC, MYC modules), the OCT4 interactome module, and the PRC2 complex revealed a pattern that correlated identically with the observed hierarchy of hiPSC reprogramming efficiencies (FIG. 6b-f). The mean expressions of MYC complex and MYC-regulated modules directly predicted the observed experimental efficiencies of hiPSC generation from the starting target populations: FL>CB>adult mPB>adult BM>keratinocytes and fibroblasts.

### Example 8

### Gene Set Enrichment Analysis (GSEA) of Primed CB Myeloid Progenitors Suggested that Factor-Driven Conversion from Hematopoietic to Pluripotent States was Accelerated by Both Soluble and Contact-Dependent Stromal Signals

The present inventors next shifted the focus toward investigating how stromal signals may augment myeloid progenitor reprogramming. A kinetic analysis of the emergence of SSEA4 and TRA antigen expression was conducted with and without BMSC co-culture during the first 4 weeks of 4F CB reprogramming (FIGS. 7a, 7b). These studies revealed that brief stromal co-culture accelerated the kinetics of factor-driven CB pluripotency induction. To determine the role of cell extrinsic paracrine vs. contact-dependent signals, CB reprogramming experiments were performed with tissue culture Transwell inserts that physically separated stromal layers from nucleofected CB cells (but allowed transfer of diffusible stromal-derived factors). These studies revealed that CB reprogramming by BMSC was enhanced by complex stromal signals that were partially contact-dependent and partially soluble factor-mediated (FIG. 7c).

To probe the identities of putative soluble factors generated by CB-BMSC interactions that augment reprogramming efficiency, supernatants from cultures of CB progenitors in the reprogramming system incubated with or without BMSCs were harvested and subjected to antibody array proteomic analysis. These studies detected the presence in the CB-BMSC secretome of multiple stem cell growth factors known to support both HSC and ESC self-renewal (e.g., BMPs, FGFs, PDGF, Wnt ligands). The top 10 stem cell growth factors identified from these secretome studies were tested for their ability to enhance CB reprogramming (FIG. 18). These studies identified PDGFbb as a potent enhancer of reprogramming efficiency. However, the present inventors did not identify one stem cell factor that could replace the need for BMSC priming, supporting the hypothesis that cell-contact stromal signals were indispensable. To gain broader insight into unique molecular pathways that might be activated by stromal priming, the global expression patterns of D3 BMSC-primed CB progenitors was evaluated by GSEA computation (FIG. 7d). This analysis revealed the activation of several unexpected contact-dependent and growth factor-mediated pathways that included ones previously implicated in potentiating ESC pluripotency such as PDGF, integrin, CCL2 chemokine, and Toll receptor signaling.

We claim:

1. A method for producing an induced pluripotent stem cell from a human myeloid progenitor cell comprising the steps of:
a. activating the human myeloid progenitor cell by incubation with hematopoietic growth factors;
b. transfecting the activated progenitor cells with an episomal plasmid expressing one or more pluripotency factors; and
c. co-culturing the transfected cells with irradiated mesenchymal bone marrow stromal cells.

2. The method of claim 1, wherein the human myeloid progenitor cell is selected from the group consisting of cord blood cell, adult bone marrow cell and adult peripheral blood cell.

3. The method of claim 1, wherein the human myeloid progenitor cell is a cord blood cell.

4. The method of claim 3, wherein the cord blood progenitor cell is CD33+CD45+.

5. The method of claim 1, wherein the hematopoietic growth factors comprise Flt3 ligand (Flt3L), stem cell factor (SCF), and thrombopoietin (TPO).

6. The method of claim 1, wherein the one or more pluripotency factors comprises sex-determining region Y HMG box 2 (SOX2), octamer binding transcription factor 4 (OCT4), Kruppel-like factor 4 (KLF4) and v-myc myelocytomatosis viral oncogene homolog (MYC).

7. The method of claim 6, wherein the one or more pluripotency factors further comprises NANOG, LIN28, and simian virus 40 large-T antigen (SV40LT).

8. The method of claim 1, wherein the one or more pluripotency factors is selected from the group consisting of SOX2, OCT4, KLF4, MYC, NANOG, LIN28, and SV40LT.

9. The method of claim 1, wherein the transfection method is nucleofection.

10. The method of claim 3, wherein the cord blood progenitor cell is CD34+CD38+.

11. A method for producing an induced pluripotent stem cell from a CD33+CD45+ cord blood progenitor cell comprising the steps of:
a. activating the cord blood progenitor cell by incubation with Flt3L, SCF and TPO;
b. nucleofecting the activated progenitor cells with an episomal plasmid expressing SOX2, OCT4, KLF4, and MYC; and
c. co-culturing the nucleofected cells with irradiated mesenchymal bone marrow stromal cells.

12. A method for producing an induced pluripotent stem cell from a growth factor activated human myeloid progenitor cell transfected with an episomal plasmid expressing one or more pluripotency factors comprising the step of co-culturing the transfected cells with irradiated mesenchymal bone marrow stromal cells following transfection.

13. The method of claim 12, wherein the human myeloid progenitor cell is selected from the group consisting of cord blood cell, adult bone marrow cell and adult peripheral blood cell.

14. The method of claim 12, wherein the human myeloid progenitor cell is a cord blood cell.

15. The method of claim 12, wherein the one or more pluripotency factors comprise SOX2, OCT4, KLF4, and MYC.

16. The method of claim 15, wherein the one or more pluripotency factors further comprises NANOG, LIN28, and SV40LT.

17. The method of claim 14, wherein the cord blood progenitor cell is CD33+CD45+.

18. The method of claim 14, wherein the cord blood progenitor cell is CD34+CD38+.

19. The method of claim 12, wherein the transfection method is nucleofection.

20. An induced pluripotent stem cell comprising an episomal plasmid encoding SOX2, OCT4, KLF4, and MYC, wherein the induced pluripotent stem cell was co-cultured with mesenchymal bone marrow stromal cells following transfection with the plasmid.

21. An induced pluripotent stem cell comprising an episomal plasmid encoding SOX2, OCT4, KLF4, MYC, NANOG, LIN28, and SV40LT, wherein the induced pluripotent stem cell was co-cultured with mesenchymal bone marrow stromal cells following transfection with the plasmid.

22. The method of claim 20 or 21, wherein the pluripotent stem cell was induced from a human myeloid progenitor cell.

23. The method of claim 22, wherein the human myeloid progenitor cell is selected from the group consisting of cord blood cell, adult bone marrow cell and adult peripheral blood cell.

24. The method of claim 20 or 21, wherein the pluripotent stem cell was induced from a cord blood progenitor cell.

25. The method of claim 24, wherein the cord blood progenitor cell is CD33+CD45+.

26. The method of claim 24, wherein the cord blood progenitor cell is CD34+CD38+.

27. The method of claim 20 or 21, wherein the transfection method is nucleofection.

28. An enriched population of isolated pluripotent stem cells produced by the method of claim 1, 11 or 12.

29. The isolated pluripotent stem cells of claim 28, wherein the isolated pluripotent stem cells express a cell surface marker selected from the group consisting of SSEA1, SSEA3, SSEA4, TRA-1-60 and TRA-1-81.

30. The isolated pluripotent stem cells of claim 28, wherein the isolated pluripotent stem cells express high embryonic stem cells (ESC)-like levels of MYC and OCT4-associated circuits and inactivated ESC-like Polycomb group (PcG)-regulated networks.

31. A method for treating a disease requiring replacement or renewal of cells comprising the step of administering to a subject an effective amount of the pluripotent stem cells of claim 1, 11 or 12.

32. A method for producing an induced pluripotent stem cell from a human myeloid progenitor cell comprising the steps of:
a. activating the human myeloid progenitor cell by incubation with hematopoietic growth factors;
b. transfecting the activated progenitor cells with a non-viral vector expressing one or more pluripotency factors; and
c. co-culturing the transfected cells with irradiated mesenchymal bone marrow stromal cells.
